(19) United States
(12) Patent Application Publication
(10) Pub. No.: US 2009/0263456 A1
(43) Pub. Date: Oct. 22, 2009

(54) Methods and Compositions for Reducing Preventing and Treating Adhesives

(75) Inventor: William McKay, Memphis, TN (US)

Correspondence Address: MEDTRONIC;Attn: Noreen Johnson - IP Legal Department, 2600 Sofamor Danek Drive, MEMPHIS, TN, 38132, US

(73) Assignee: WARSAW ORTHOPEDIC, INC., Warsaw, IN (US)

(21) Appl. No.: 12/106,038

(22) Filed: Apr. 18, 2008

(51) Int. Cl. A61K 9/00; A61P 41/00

(52) U.S. Cl. 424/426

(57) ABSTRACT

Methods and compositions are provided for reducing, treating or preventing adhesions in a patient in need of such treatment, the methods and compositions comprising administering one or more biodegradable drug depots comprising a therapeutically effective amount of a glucocorticoid or pharmaceutically acceptable salt thereof to a target tissue site beneath the skin, wherein the drug depot releases an effective amount of the glucocorticoid or pharmaceutically acceptable salt thereof to prevent in-growth of scar tissue and formation or reformation of adhesions at or near the target tissue site while an injured tissue heals.

### BACKGROUND

Surgical adhesions are abnormal fibrous bands of scar tissue that can form inside the body as a result of the healing process that often follows open or minimally invasive surgical procedure including abdominal, gynecologic, cardiothoracic, spinal, plastic, vascular, ENT, ophthalmologic, urologic, neuro, or orthopedic surgery.

Surgical adhesions are typically connective tissue structures that form between adjacent injured areas within the body. Briefly, localized areas of injury trigger an inflammatory and healing response (clotting) that culminates in healing and scar tissue formation. If scarring results in the formation of fibrous tissue bands or adherence of adjacent anatomical structures (that should normally be separate), adhesion formation is said to have occurred.

Adhesions can range from flimsy, easily separable structures to dense, tenacious fibrous structures that can only be separated by surgical dissection. While many adhesions are benign, many adhesions can cause major pain. For example, after spinal surgery if adhesions form they may cause tethering of spinal nerve roots and dorsal root ganglia, which often causes recurrent radicular pain that can be very debilitating to the patient and often leads to repeat surgical intervention.

Since most surgery involves a certain degree of trauma to the operative tissues, virtually any procedure (no matter how well executed) has the potential to result in the formation of clinically significant adhesion formation. Adhesions can be triggered by surgical trauma such as cutting, manipulation, retraction or suturing, as well as from inflammation, infection (e.g., fungal or mycobacterium), bleeding or the presence of a foreign body. Surgical trauma may also result from tissue drying, ischemia, or thermal injury. Due to the diverse etiology of surgical adhesions, the potential for formation exists regardless of whether the surgery is done in a so-called minimally invasive fashion (e.g., catheter-based therapies, laparoscopy) or in a standard open technique involving one or more relatively large incisions. Although a potential complication of any surgical intervention, surgical adhesions are particularly problematic in GI surgery (causing bowel obstruction), gynecological surgery (causing pain and/or infertility), tendon repairs (causing shortening and flexion deformities), joint capsule procedures (causing capsular contractures), and nerve and muscle repair procedures (causing diminished or lost function).

Surgical adhesions may cause various, often serious and unpredictable clinical complications; some of which manifest themselves only years after the original procedure was completed. Complications from surgical adhesions are a major cause of failed surgical therapy and are the leading cause of bowel obstruction and infertility. Other adhesion-related complications include chronic back or pelvic pain, intestinal obstruction, urethral obstruction and voiding dysfunction.

Relieving the post-surgical complications caused by adhesions generally requires another surgery. However, the subsequent surgery is further complicated by adhesions formed as a result of the previous surgery. In addition, the second surgery is likely to result in further adhesions and a continuing cycle of additional surgical complications.

Thus, new compositions and methods are needed to reduce, prevent, or treat adhesions. New compositions and methods that reliably prevent in-growth of scar tissue and formation or reformation of adhesions at or near the target tissue site while an injured tissue heals are needed.

### SUMMARY

New compositions and methods are provided that effectively reduce, prevent or treat adhesions. In various embodiments, glucocorticoid compositions and methods are provided that have anti-adhesion effects in a single drug depot or multiple drug depots. New glucocorticoid compositions and methods are provided, which can easily allow accurate and precise implantation of a drug depot containing the glucocorticoid with minimal physical and psychological trauma to a patient. One advantage of the glucocorticoid drug depot compositions and methods is that the drug depot can now be easily delivered to the target tissue site (e.g., abdomen, synovial joint, at or near the spinal column, etc.) and reduce, prevent or treat adhesion formation. In this way, accurate and precise implantation of the drug depot in a minimally invasive procedure can be accomplished.

In one embodiment, a method of reducing, preventing or treating adhesions in a patient in need of such treatment is provided, the method comprising administering one or more biodegradable drug depots comprising a therapeutically effective amount of a glucocorticoid or a pharmaceutically acceptable salt thereof at or near a target tissue site beneath the skin, wherein the one or more biodegradable drug depot is capable of releasing an effective amount of the glucocorticoid or pharmaceutically acceptable salt thereof over a period of at least one day to 6 months.

In another embodiment, a method is provided that utilizes one or more drug depots that release an effective amount of the fluocinolone or dexamethasone or a pharmaceutically acceptable salt thereof over a period of at least 1 week to 6 weeks to reduce, prevent or inhibit adhesions.

In yet another embodiment, a barrier is administered before, after or with the one or more drug depots at or near the target tissue site to reduce, prevent or inhibit adhesions.

In one exemplary embodiment, an implantable drug depot useful for reducing, preventing or treating adhesions in a patient in need of such treatment is provided, the implantable drug depot comprising a therapeutically effective amount of a glucocorticoid or a pharmaceutically acceptable salt thereof, the depot being implantable at a site beneath the skin to reduce, prevent or treat adhesions, wherein the drug depot is loaded with about 0.5 weight % to about 40 weight % of the glucocorticoid or a pharmaceutically acceptable salt thereof and is capable of releasing an effective amount of a glucocorticoid or pharmaceutically acceptable salt thereof over a period of at least 1 week to 6 weeks.

In another exemplary embodiment, a method of making an implantable drug depot is provided, the method comprising combining a biocompatible polymer and a therapeutically effective amount of a glucocorticoid or a pharmaceutically acceptable salt thereof and forming the implantable drug depot from the combination.

Additional features and advantages of various embodiments will be set forth in part in the description that follows, and in part will be apparent from the description, or may be learned by practice of various embodiments. The objectives and other advantages of various embodiments will be realized and attained by means of the elements and combinations particularly pointed out in the description and appended claims.

### BRIEF DESCRIPTION OF THE DRAWINGS

In part, other aspects, features, benefits and advantages of the embodiments will be apparent with regard to the following description, appended claims and accompanying drawings where:

FIG. 1 illustrates a number of common locations within a patient that may be sites where surgery is conducted and locations where the drug depot containing a glucocorticoid can locally be administered thereto.

FIG. 2 illustrates a schematic dorsal view of the spine and sites where a drug depot containing a glucocorticoid can locally be administered thereto.

FIGS. 3 and 4 show pictures of the surgical wound near the common sciatic nerve of a rat where a drug depot (containing 20 wt % fluocinolone, 70 wt % 85/15 PLGA, and 10 wt % mPEG) was implanted. No noticeable adhesions were noted after 57 days of treatment, little to no encapsulation and sutures appear relatively intact.

FIG. 5 shows a picture of the surgical wound near the common sciatic nerve of a rat where a drug depot (containing the NSAID sulindac) was implanted. Adhesions formed indicated by the scar tissue and fibrous tissue.

FIG. 6 shows a picture of the surgical wound near the common sciatic nerve of a rat where a drug depot containing 1 wt. % fluocinolone, 100 DL 7E+5% PEG/7% 5050 was implanted. No noticeable adhesion formation after 57 days of treatment and little to no encapsulation.

FIG. 7 shows the various fluocinolone drug depots made including drug loads, polymers and excipients.

FIGS. 8-9 are graphic representations of in vitro % cumulative release of fluocinolone over periods of time ranging from about 20 days to about 100 days with fluocinolone release rates starting at 0% to 60% cumulative release.

It is to be understood that the figures are not drawn to scale. Further, the relation between objects in a figure may not be to scale, and may in fact have a reverse relationship as to size. The figures are intended to bring understanding and clarity to the structure of each object shown, and thus, some features may be exaggerated in order to illustrate a specific feature of a structure.

### DETAILED DESCRIPTION

For the purposes of this specification and appended claims, unless otherwise indicated, all numbers expressing quantities of ingredients, percentages or proportions of materials, reaction conditions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth, the broad scope of the invention are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges subsumed therein. For example, a range of “1 to 10” includes any and all subranges between (and including) the minimum value of 1 and the maximum value of 10, that is, any and all subranges having a minimum value of equal to or greater than 1 and a maximum value of equal to or less than 10, e.g., 5.5 to 10.

It is noted that, as used in this specification and the appended claims, the singular forms “a,” “an,” and “the,” include plural referents unless expressly and unequivocally limited to one referent. Thus, for example, reference to “a drug depot” includes one, two, three or more drug depots.

Reference will now be made in detail to certain embodiments of the invention, examples of which are illustrated in the accompanying drawings. While the invention will be described in conjunction with the illustrated embodiments, it will be understood that they are not intended to limit the invention to those embodiments. On the contrary, the invention is intended to cover all alternatives, modifications, and equivalents, which may be included within the invention as defined by the appended claims.

The headings below are not meant to limit the disclosure in any way; embodiments under any one heading may be used in conjunction with embodiments under any other heading.

New compositions and methods are provided that effectively reduce, prevent or treat adhesions. In various embodiments, glucocorticoid compositions and methods are provided that have anti-adhesion effects in a single drug depot or multiple drug depots. New glucocorticoid compositions and methods are provided, which can easily allow accurate and precise implantation of a drug depot containing the glucocorticoid with minimal physical and psychological trauma to a patient. One advantage of the glucocorticoid drug depot compositions and methods is that the drug depot can now be easily delivered to the target tissue site (e.g., abdomen, synovial joint, at or near the spinal column, etc.) and reduce, prevent or treat adhesion formation. In this way, accurate and precise implantation of the drug depot in a minimally invasive procedure can be accomplished.

In one embodiment, a method of reducing, preventing or treating adhesions in a patient in need of such treatment is provided, the method comprising administering one or more biodegradable drug depots comprising a therapeutically effective amount of a glucocorticoid or a pharmaceutically acceptable salt thereof at or near a target tissue site beneath the skin, wherein the one or more biodegradable drug depot is capable of releasing an effective amount of the glucocorticoid or pharmaceutically acceptable salt thereof over a period of at least 1 day to 6 months.

In another embodiment, a method is provided that utilizes one or more drug depots that release an effective amount of the fluocinolone or dexamethasone or a pharmaceutically acceptable salt thereof over a period of at least 1 week to 6 weeks to reduce, prevent or inhibit adhesions.

### Glucocorticoids

A glucocorticoid is contained in a drug depot. A drug depot comprises a physical structure to facilitate sustained release of the drug in a desired site (e.g., a synovial joint, a disc space, a spinal canal, abdominal area, a tissue of the patient, etc.). The drug depot also comprises the drug. The term “drug” as used herein is generally meant to refer to any substance that alters the physiology of a the patient. The term “drug” may be used interchangeably herein with the terms “therapeutic agent”, “therapeutically effective amount”, and “active pharmaceutical ingredient” or “API”. It will be understood that a “drug” formulation may include more than one therapeutic agent, wherein exemplary combinations of therapeutic agents include a combination of two or more drugs. The drug depot provides a concentration gradient of the therapeutic agent around the depot for delivery to the site. In various embodiments, the drug depot provides an optimal drug concentration gradient of the therapeutic agent at a distance of up to about 0.1 cm to about 5 cm from the implant site.

A “therapeutically effective amount” or “effective amount” is such that when administered, the drug results in alteration of the biological activity, such as, for example, inhibition of inflammation, reduction or alleviation of pain, improvement in the condition, etc. In various embodiments, the therapeutically effective amount of a glucocorticoids is that amount that prevents, reduces or treats adhesions. It will be understood that the dosage administered to a patient can be as single depot or multiple depots depending upon a variety of factors, including the drug's administered pharmacokinetic properties, the route of administration, patient conditions and characteristics (sex, age, body weight, health, size, etc.), extent of symptoms, concurrent treatments, frequency of treatment and the effect desired.

Glucocorticoids are a class of steroids characterized by an ability to bind with the glucocorticoid receptor. Glucocorticoids have a broad spectrum of anti-inflammatory and immunosuppressive effects. They act by inhibiting leukocyte traffic; interfering with functions of leukocytes, fibroblasts, and endothelial cells; and suppressing the synthesis and actions of inflammatory cytokines including interleukin-6. Glucocorticoids affect glucose metabolism. The glucocorticoids used herein have at least some glucocorticoid activity and optionally may have some mineralocorticoid activity.

As used herein “glucocorticoid” encompasses a glucocorticoid or pharmaceutically acceptable salts thereof; pharmacologically-active derivatives of the glucocorticoid or an active metabolite of the glucocorticoid. As used herein, “pharmaceutically acceptable salts” refer to derivatives of the disclosed compounds (e.g., esters or amines) wherein the parent compound may be modified by making acidic or basic salts thereof. Examples of pharmaceutically acceptable salts include, but are not limited to, mineral or organic acid salts of basic residues such as amines; alkali or organic salts of acidic residues such as carboxylic acids. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, or nitric acids; or the salts prepared from organic acids such as acetic, fuoric, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, tolunesulfonic, methanesulfonic, ethane disulfonic, oxalic, isethionic acid. Pharmaceutically acceptable also includes the racemic mixtures ((+)-R and (−)-S enantiomers) or each of the dextro and levo isomers of the glucocorticoid individually. The glucocorticoids may be in the free acid or base form or be pegylated for long acting activity.

A suitable glucocorticoid, includes but is not limited to, alclometasone, aldosterone amcinonide, 21-acetoxypregnenolone, algestone, amcinonide, beclomethasone, betamethasone, budesonide, beclometasone, budesonide, ciclesonide, clobetasol, clobetasone, clocortolone, cloprednol, cortivazol, chloroprednisone, corticosterone, cortisone, deflazacort, deoxycorticosterone, desonide, desoxycortone, desoximetasone, dexamethasone, dexamethasone acetate, dexamethasone phosphate di-sodium salt, diflorasone, diflucortolone, difluprednate, enoxolone, fluazacort, flumethasone, flunisolide, fluocinolone, fluocinolone acetonide, fluperolone acetate, fluprednidene acetate, fluprednisolone, flurandrenolide, fluticasone propionate, fluclorolone, fludrocortisone, fludroxycortide, flumetasone, flunisolide, fluocinonide, fluocortin, fluocortolone, fluorometholone, fluperolone, fluprednidene, fluticasone, formocortal, halcinonide, halometasone, halobetasol propionate, halopredone acetate, hydrocortamate, hydrocortisone, hydrocortisone aceponate, hydrocortisone buteprate, hydrocortisone butyrate, loteprednol, loteprednol etabonate, mazipredone, medrysone, meprednisone, mometasone furoate, medrysone, meprednisone, methylprednisolone, methylprednisolone aceponate, mometasone furoate, paramethasone, prednicarbate, prednisone, prednisolone, prednylidene, paramethasone, prednicarbate, prednisolone 25-diethylamino-acetate, prednisolone sodium phosphate, prednisone, prednival, prednylidene, rimexolone, tixocortol, triamcinolone, triamcinolone acetonide, triamcinolone benetonide, triamcinolone hexacetonide, ulobetasol or pharmaceutically acceptable salts or esters or amines thereof or a combination thereof.

Glucocorticoids are distinguished from mineralocorticoids and sex steroids by their specific receptors, target cells, and effects. For example, on one hand, mineralocorticoids exert their effect on the kidneys, causing selective excretion of excess potassium in the urine and at the same time conservation and/or retention of sodium. On the other hand, sex steroids such as the female hormones estrogen and progesterone and the male androgens such as testosterone are used for male/female development.

In one exemplary embodiment, to prevent, reduce or treat adhesions, the glucocorticoid comprises fluocinolone or a pharmaceutically acceptable salt thereof. Some examples of potentially pharmaceutically acceptable salts include those salt-forming acids and bases that do not substantially increase the toxicity of the compound, such as, salts of alkali metals such as magnesium, potassium and ammonium, salts of mineral acids such as hydrochloric, hydriodic, hydrobromic, phosphoric, metaphosphoric, nitric and sulfuric acids, as well as salts of organic acids such as tartaric, acetic, citric, malic, benzoic, glycollic, gluconic, gulonic, succinic, arylsulfonic, e.g., p-toluenesulfonic acids, and the like. To the extent that salts of fluocinolone can be created for safe administration to a mammal, they are within the scope of the application herein. Further, when referring to fluocinolone the active ingredient may not only be in the salt form, but also in the base form (e.g., free acid), amine, ester or racemic forms or a combination thereof.

### Fluocinolone

Fluocinolone is available from various pharmaceutical manufacturers. In various embodiments, the fluocinolone comprises fluocinolone acetonide. The dosage of fluocinolone may be from approximately 0.0005 to approximately 100 μg/kg/day. Additional dosages of fluocinolone include from approximately 0.0005 to approximately 95 μg/kg/day; approximately 0.0005 to approximately 90 μg/kg/day; approximately 0.0005 to approximately 85 μg/kg/day; approximately 0.0005 to approximately 80 μg/kg/day; approximately 0.0005 to approximately 75 μg/kg/day; approximately 0.001 to approximately 70 μg/kg/day; approximately 0.001 to approximately 65 μg/kg/day; approximately 0.001 to approximately 60 μg/kg/day; approximately 0.001 to approximately 55 μg/kg/day; approximately 0.001 to approximately 50 μg/kg/day; approximately 0.001 to approximately 45 μg/kg/day; approximately 0.001 to approximately 40 μg/kg/day; approximately 0.001 to approximately 35 μg/kg/day; approximately 0.0025 to approximately 30 μg/kg/day; approximately 0.0025 to approximately 25 μg/kg/day; approximately 0.0025 to approximately 20 μg/kg/day; and approximately 0.0025 to approximately 15 μg/kg/day. In another embodiment, the dosage of fluocinolone is from approximately 0.005 to approximately 15 μg/kg/day. In another embodiment, the dosage of fluocinolone is from approximately 0.005 to approximately 10 μg/kg/day. In another embodiment, the dosage of fluocinolone is from approximately 0.005 to approximately 5 μg/kg/day. In another embodiment, the dosage of fluocinolone is from approximately 0.005 to 2.5 μ/kg/day. In some embodiments, the amount of fluocinolone is between 40 and 600 μg/day. In some embodiments, the amount of fluocinolone is between 200 and 400 μg/day. In various embodiments, the fluocinolone load in one or more drug depots can be 0.5 wt. % to 20 wt. %.

### Dexamethasone

In one exemplary embodiment, to prevent, reduce or treat adhesions, the glucocorticoid comprises dexamethasone or a pharmaceutically acceptable salt thereof. When referring to dexamethasone, unless otherwise specified or apparent from context it is understood that the inventors are also referring to pharmaceutically acceptable salts. Some examples of potentially pharmaceutically acceptable salts include those salt-forming acids and bases that do not substantially increase the toxicity of the compound, such as, salts of alkali metals such as magnesium, potassium and ammonium, salts of mineral acids such as hydrochloric, hydriodic, hydrobromic, phosphoric, metaphosphoric, nitric and sulfuric acids, as well as salts of organic acids such as tartaric, acetic, citric, malic, benzoic, glycollic, gluconic, gulonic, succinic, arylsulfonic, e.g., p-toluenesulfonic acids, and the like. To the extent that salts of dexamethasone can be created for safe administration to a mammal, they are within the scope of the present application. When referring to dexamethasone unless otherwise specified, the specification also includes dexamethasone acetate and/or dexamethasone sodium phosphate.

Further, when referring to dexamethasone the active ingredient may not only be in the salt form, but also in the base form (e.g., free acid). In various embodiments, if it is in the acid form, it may be combined with polymers under conditions in which there is not severe polymer degradation, as may be seen upon heat or solvent processing that may occur with PLGA or PLA. In various embodiments, the drug depot comprises from about 5 wt. % to 20 wt. % dexamethasone acetate and the polymer comprises 75/25 or 85/25 PLGA, POE, or SAIB, with or without mPEG.

Dexamethasone is available from various manufacturers. In various embodiments, dexamethasone may be released from the depot at a dose of about 10 pg to about 10 mg/hr, about 100 pg/hr to about 1 mg/hr, about 1 ng/hr to about 100 ug/hr, about 10 ng/hr to about 10 ug/hr, about 100 ng/hr to about 1 ug/hr or about 500 ug/hr. In various embodiments, the dose may be about 0.01 to about 10 mg/kg/day or about 1 mg to about 120 mg/day.

In addition to the glucocorticoids, the drug depot may comprise one or more additional therapeutic agents. Examples of therapeutic agents include, those that are direct- and local-acting modulators of pro-inflammatory cytokines such as TNF-α and IL-1 including, but not limited to, soluble tumor necrosis factor α receptors, any pegylated soluble tumor necrosis factor α receptor, monoclonal or polyclonal antibodies or antibody fragments or combinations thereof. Examples of suitable therapeutic agents include receptor antagonists, molecules that compete with the receptor for binding to the target molecule, antisense polynucleotides, and inhibitors of transcription of the DNA encoding the target protein. Suitable examples include, but are not limited to, Adalimumab, Infliximab, Etanercept, Pegsunercept (PEG sTNF-R1), sTNF-R1, CDP-870, CDP-571, CNI-1493, RDP58, ISIS 104838, 1→3-β-D-glucans, Lenercept, PEG-sTNFRII Fc Mutein, D2E7, Afelimomab, and combinations thereof. In other embodiments, a therapeutic agent includes metalloprotease inhibitors, glutamate antagonists, glial cell-derived neurotropic factors (GDNF), B2 receptor antagonists, Substance P receptor (NK1) antagonists such as capsaicin and civamide, downstream regulatory element antagonistic modulator (DREAM), iNOS, inhibitors of tetrodotoxin (TTX)-resistant Na+-channel receptor subtypes PN3 and SNS2, inhibitors of interleukins such as IL-1, IL-6 and IL-8, and anti-inflammatory cytokines, TNF binding protein, onercept (r-hTBP-1), recombinant adeno-associated viral (rAAV) vectors encoding inhibitors, enhancers, potentiators, or neutralizers, antibodies, including, but not limited to, naturally occurring or synthetic, double-chain, single-chain, or fragments thereof. For example, suitable therapeutic agents include molecules that are based on single chain antibodies called Nanobodies™ (Ablynx, Ghent Belgium), which are defined as the smallest functional fragment of a naturally occurring, single-domain antibody. Alternatively, therapeutic agents include agents that effect kinases and/or inhibit cell signaling mitogen-activated protein kinases (MAPK), p38 MAPK, Src or protein tyrosine kinase (PTK). Therapeutic agents include, kinase inhibitors such as, for example, Gleevec, Herceptin, Iressa, imatinib (STI571), herbimycin A, tyrphostin 47, erbstatin, genistein, staurosporine, PD98059, SB203580, CNI-1493, VX-50/702 (Vertex/Kissei), SB203580, BIRB 796 (Boehringer Ingelheim), Glaxo P38 MAP Kinase inhibitor, RWJ67657 (J&J), UO126, Gd, SCIO-469 (Scios), RO3201195 (Roche), Semipimod (Cytokine PharmaSciences), or derivatives thereof.

Therapeutic agents, in various embodiments, block the transcription or translation of TNF-α or other proteins in the inflammation cascade. Suitable therapeutic agents include, but are not limited to, integrin antagonists, alpha-4 beta-7 integrin antagonists, cell adhesion inhibitors, interferon gamma antagonists, CTLA4-Ig agonists/antagonists (BMS-188667), CD40 ligand antagonists, Humanized anti-IL-6 mAb (MRA, Tocilizumab, Chugai), HMGB-1 mAb (Critical Therapeutics Inc.), anti-IL2R antibodies (daclizumab, basilicimab), ABX (anti IL-8 antibodies), recombinant human IL-10, or HuMax IL-15 (anti-IL 15 antibodies).

Other suitable therapeutic agents include IL-1 inhibitors, such Kineret® (anakinra) which is a recombinant, non-glycosylated form of the human inerleukin-1 receptor antagonist (IL-1Ra), or AMG 108, which is a monoclonal antibody that blocks the action of IL-1. Therapeutic agents also include excitatory amino acids such as glutamate and aspartate, antagonists or inhibitors of glutamate binding to NMDA receptors, AMPA receptors, and/or kainate receptors. Interleukin-1 receptor antagonists, thalidomide (a TNF-α release inhibitor), thalidomide analogues (which reduce TNF-α production by macrophages), bone morphogenetic protein (BMP) type 2 and BMP-4 (inhibitors of caspase 8, a TNF-α activator), quinapril (an inhibitor of angiotensin II, which upregulates TNF-α), interferons such as IL-11 (which modulate TNF-α receptor expression), and aurin-tricarboxylic acid (which inhibits TNF-α), for example, may also be useful as therapeutic agents for reducing inflammation. It is contemplated that where desirable a pegylated form of the above may be used. Examples of other therapeutic agents include NF kappa B inhibitors such as, clonidine; antioxidants, such as dilhiocarbamate, and other compounds, such as, for example, bupivacaine, or sulfasalazine.

Specific examples of therapeutic agents suitable for use include, but are not limited to an anti-inflammatory agent, analgesic agent, or osteoinductive growth factor or a combination thereof. Anti-inflammatory agents include, but are not limited to, salicylates, diflunisal, sulfasalazine, indomethacin, ibuprofen, ketorolac, naproxen, tolmetin, diclofenac, ketoprofen, fenamates (mefenamic acid, meclofenamic acid), enolic acids (piroxicam, meloxicam), celecoxib, etodolac, nimesulide, apazone, sulindac or tepoxalin; antioxidants, such as dithiocarbamate, or other compounds such as sulfasalazine [2-hydroxy-5-[-4-[C2-pyridinylamino)sulfonyl]azo]benzoic acid] or a combination thereof.

Suitable anabolic growth or anti-catabolic growth factors include, but are not limited to, a bone morphogenetic protein, a growth differentiation factor, a LIM mineralization protein, CDMP or progenitor cells or a combination thereof.

Suitable analgesic agents include, but are not limited to, acetaminophen, lidocaine, bupivicaine, opioid analgesics such as buprenorphine, butorphanol, dextromoramide, dezocine, dextropropoxyphene, diamorphine, fentanyl, alfentanil, sufentanil, hydrocodone, hydromorphone, ketobemidone, levomethadyl, mepiridine, methadone, morphine, nalbuphine, opium, oxycodone, papaveretum, pentazocine, pethidine, phenoperidine, piritramide, dextropropoxyphene, remifentanil, tilidine, tramadol, codeine, dihydrocodeine, meptazinol, dezocine, eptazocine, flupirtine or a combination thereof.

Analgesics also include agents with analgesic properties, such as for example, amitriptyline, carbamazepine, gabapentin, pregabalin, clonidine, or a combination thereof.

The depot may contain a muscle relaxant. Exemplary muscle relaxants include by way of example and not limitation, alcuronium chloride, atracurium bescylate, baclofen, carbolonium, carisoprodol, chlorphenesin carbamate, cyclobenzaprine, dantrolene, decamethonium bromide, fazadinium, gallamine triethiodide, hexafluorenium, meladrazine, mephensin, metaxalone, methocarbamol, metocurine iodide, pancuronium, pridinol mesylate, styramate, suxamethonium, suxethonium, thiocolchicoside, tizanidine, tolperisone, tubocuarine, vecuronium, or combinations thereof.

The depot comprises the therapeutic agent or agents and may also contain other non-active ingredients or excipients. It has a multi-functional purpose including the carrying, stabilizing and controlling the release of the therapeutic agent(s). The controlled release process, for example, may be by a solution-diffusion mechanism or it may be governed by an erosion-controlled process. Typically, the depot will be a solid or semi-solid formulation comprised of a biocompatible material, which can be biodegradable. The term “solid” is intended to mean a rigid material, while, “semi-solid” is intended to mean a material that has some degree of flexibility, thereby allowing the depot to bend and conform to the surrounding tissue requirements. Some examples of excipients include, for example, mPEG (methoxypolyethyleneglycol), sorbitol, D-sorbitol, maltodextrin, cyclodextrin, B-cyclodextrin, or combinations thereof. The excipients may be added in weight percentages from 0.5% to 50%.

In various embodiments, the depot material will be durable within the tissue site for a period of time equal to (for biodegradable components) or greater than (for non-biodegradable components) the planned period of drug delivery. For example, the depot material may have a melting point or glass transition temperature close to or higher than body temperature, but lower then the decomposition or degradation temperature of the therapeutic agent. However, the pre-determined erosion of the depot material can also be used to provide for slow release of the loaded therapeutic agent(s).

In various embodiments, the drug depot may be designed to release the glucocorticoid when certain trigger points are reached (e.g., temperature, pH, etc.) after implantation in vivo. For example, the drug depot may comprise polymers that will release more drug as the body temperature reaches greater than, for example, 102° F., particularly if the drug possesses antipyretic properties such as a glucocorticoid. In various embodiments, depending on the site of implantation, the drug depot may release more or less drug as a certain pH is reached. For example, the drug depot may be designed to release the drug as the bodily fluid having a certain pH contact the drug depot (e.g., CSF having a pH of about 7.35 to about 7.70, synovial fluid having a pH of about 7.29 to about 7.45; urine having a pH of about 4.6 to about 8.0, pleural fluids having a pH of about 7.2 to about 7.4, blood having a pH of about 7.35 to about 7.45, etc.)

In various embodiments, the depot may have a high drug loading, such that the glucocorticoid and/or other therapeutic agent comprises about 20-99 wt % of the depot, or 20-95 wt % of the depot, or 50-95 wt % of the depot. In various embodiments, the amount of glucocorticoid and/or other therapeutic agent are present in the depot in a range from about 0.1% to about 40% by weight of the depot (including 0.1%, 0.2%, 0.5%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, and ranges between any two of these points, for instance, 0.1-10%, 10-20% and 20-30%, etc.). In various embodiments, the glucocorticoids can be used in a load range of 2-20%.

In one exemplary embodiment, with drug loads of 1% to 20% fluocinolone, 85/15 PLGA or DL-PLA or DL-PLA and 50/50 PLGA mixture can be added in an amount of from about 10% to 98%.

In various embodiments, the drug depot comprises poly(lactide-co-glycolide) (PLGA), polylactide (PLA), polyglycolide (PGA), D-lactide, D,L-lactide, L-lactide, D,L-lactide-ε-caprolactone, D,L-lactide-glycolide-ε-caprolactone, glycolide-caprolactone or a combination thereof.

In one exemplary embodiment, with drug loads of 5% to 20% dexamethasone base or acetate, 85/15 PLGA or 75/25 PLGA, or POE, or SAIB can be added in an amount of from about 10% to 98%. As persons of ordinary skill in the art are aware, implantable elastomeric depot compositions having a blend of polymers with different end groups are used the resulting formulation will have a lower burst index and a regulated duration of delivery. For example, one may use polymers with acid (e.g., carboxylic acid) and ester end groups (e.g., lauryl, methyl or ethyl ester end groups).

In various embodiments, the drug depot may release approximately 0.005 to approximately 10 μg/kg/day of a glucocorticoid for a total of at least one day to 6 months, or 1 to 8 weeks or 2 to 6 weeks to reduce, prevent, or treat adhesions.

In various embodiments, the drug depot releases 5%, 10%, 15%, 20%, 25%, 30%, 40%, 50%, 60%, 70%, 80%, 90%, 95%, or 99% of a glucocorticoid over a period of 1 to 8 weeks or 2 to 6 weeks after the drug depot is administered to the target tissue site to reduce, prevent or treat adhesions. The drug depot may have a “release rate profile” that refers to the percentage of active ingredient that is released over fixed units of time, e.g., mcg/hr, mg/hr, mcg/day, mg/day, 10% per day for one week, ten days, etc. As persons of ordinary skill know a release rate profile may be but need not be linear.

In some embodiments, the drug depot may not be biodegradable. For example, the drug depot may comprise polyurethane, polyurea, polyether(amide), PEBA, thermoplastic elastomeric olefin, copolyester, and styrenic thermoplastic elastomer, steel, aluminum, stainless steel, titanium, metal alloys with high non-ferrous metal content and a low relative proportion of iron, carbon fiber, glass fiber, plastics, ceramics or combinations thereof. Typically, these types of drug depots may need to be removed after a certain amount of time.

In some instances, it may be desirable to avoid having to remove the drug depot after use. In those instances, the depot may comprise a biodegradable material. There are numerous materials available for this purpose and having the characteristic of being able to breakdown or disintegrate over a prolonged period of time when positioned at or near the target tissue. As function of the chemistry of the biodegradable material the mechanism of the degradation process can be hydrolytical or enzymatical in nature, or both. In various embodiments, the degradation can occur either at the surface (heterogeneous or surface erosion) or uniformly throughout the drug delivery system depot (homogeneous or bulk erosion).

A “depot” includes, but is not limited to, capsules, microspheres, microparticles, microcapsules, microfibers particles, nanospheres, nanoparticles, coating, matrices, wafers, pills, pellets, emulsions, liposomes, micelles, gels, or other pharmaceutical delivery compositions. Suitable materials for the depot are ideally pharmaceutically acceptable biodegradable and/or any bioabsorbable materials that are preferably FDA approved or GRAS materials. These materials can be polymeric or non-polymeric, as well as synthetic or naturally occurring, or a combination thereof.

The term “biodegradable” includes that all or parts of the drug depot will degrade over time by the action of enzymes, by hydrolytic action and/or by other similar mechanisms in the human body. In various embodiments, “biodegradable” includes that depot (e.g., microparticle, microsphere, gel, etc.) can break down or degrade within the body to non-toxic components after or while a therapeutic agent has been or is being released. By “bioerodible” it is meant that the depot and/or gel will erode or degrade over time due, at least in part, to contact with substances found in the surrounding tissue, fluids or by cellular action. By “bioabsorbable” it is meant that the depot will be broken down and absorbed within the human body, for example, by a cell or tissue. “Biocompatible” means that the depot will not cause substantial tissue irritation or necrosis at the target tissue site.

In various embodiments, the depot may comprise a bioabsorbable, bioerodible, and/or a biodegradable biopolymer that may provide immediate release, sustained release or controlled release of the drug. Examples of suitable sustained release biopolymers include, but are not limited to, poly(alpha-hydroxy acids), poly(lactide-co-glycolide) (PLGA), polylactide (PLA), polyglycolide (PG), polyethylene glycol (PEG), PEG 200, PEG 300, PEG 400, PEG 500, PEG 550, PEG 600, PEG 700, PEG 800, PEG 900, PEG 1000, PEG 1450, PEG 3350, PEG 4500, PEG 8000, conjugates of poly(alpha-hydroxy acids), polyorthoesters, polyaspirins, polyphosphagenes, collagen, starch, pre-gelatinized starch, hyaluronic acid, chitosans, gelatin, alginates, albumin, fibrin, vitamin E analogs, such as alpha tocopheryl acetate, d-alpha tocopheryl succinate, D,L-lactide, or L-lactide, -caprolactone, dextrans, vinylpyrrolidone, polyvinyl alcohol (PVA), PVA-g-PLGA, PEGT-PBT copolymer (polyactive), methacrylates, poly(N-isopropylacrylamide), PEO-PPO-PEO (pluronics), PEO-PPO-PAA copolymers, PLGA-PEO-PLGA, PEG-PLG, PLA-PLGA, poloxamer 407, PEG-PLGA-PEG triblock copolymers, SAIB (sucrose acetate isobutyrate) hydroxypropyl cellulose, hydroxypropyl methylcellulose, hydroxyethyl methylcellulose, carboxymethylcellulose or salts thereof, Carbopol, poly(hydroxyethylmethacrylate), poly(methoxyethylmethacrylate), poly(methoxyethoxy-ethylmethacrylate), polymethylmethacrylate (PMMA), methylmethacrylate (MMA), gelatin, polyvinyl alcohols, propylene glycol, or combinations thereof.

In various embodiments, when the drug depot comprises a polymer, it may be employed at about 10 wt % to about 99 wt % or about 30 wt % to about 60 wt % based on the weight of the drug depot.

The depot may optionally contain inactive materials such as buffering agents and pH adjusting agents such as potassium bicarbonate, potassium carbonate, potassium hydroxide, sodium acetate, sodium borate, sodium bicarbonate, sodium carbonate, sodium hydroxide or sodium phosphate; degradation/release modifiers; drug release adjusting agents; emulsifiers; preservatives such as benzalkonium chloride, chlorobutanol, phenylmercuric acetate and phenylmercuric nitrate, sodium bisulfite, sodium bisulfate, sodium thiosulfate, thimerosal, methylparaben, polyvinyl alcohol and phenylethyl alcohol; solubility adjusting agents; stabilizers; and/or cohesion modifiers. Typically, any such inactive materials will be present within the range of 0-75 wt %, and more typically within the range of 0-30 wt %. If the depot is to be placed in the spinal area or joint area, in various embodiments, the depot may comprise sterile preservative free material.

The depot can be different sizes, shapes and configurations. There are several factors that can be taken into consideration in determining the size, shape and configuration of the drug depot. For example, both the size and shape may allow for ease in positioning the drug depot at the target tissue site that is selected as the implantation or injection site. In addition, the shape and size of the system should be selected so as to minimize or prevent the drug depot from moving after implantation or injection. In various embodiments, the drug depot can be shaped like a sphere, a cylinder such as a rod or pellet, fiber, a flat surface such as a disc, film, or sheet, or the like. Flexibility may be a consideration so as to facilitate placement of the drug depot. In various embodiments, the drug depot can be different sizes, for example, the drug depot may be a length of from about 0.5 mm to 5 mm and have a diameter of from about 0.01 to about 2 mm. In various embodiments, the drug depot may have a layer thickness of from about 0.005 to 1.0 mm, such as, for example, from 0.05 to 0.75 mm.

Radiographic markers can be included on the drug depot to permit the user to accurately position the depot into the target site of the patient. These radiographic markers will also permit the user to track movement and degradation of the depot at the site over time. In this embodiment, the user may accurately position the depot in the site using any of the numerous diagnostic imaging procedures. Such diagnostic imaging procedures include, for example, X-ray imaging or fluoroscopy. Examples of such radiographic markers include, but are not limited to, barium, calcium, and/or metal beads or particles. Where present, the radiographic marker is typically present in an amount of from about 10% to about 40% (including 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39% and 40%, as well as ranges between any two of these values, e.g., 10-15%, 15-20%, 20-25%, 25-30%, 30-35%, 35-40%, and so forth, with 15-30% being more typical, even more typically 20-25%). In various embodiments, the radiographic marker could be a spherical shape or a ring around the depot.

In one exemplary embodiment, a drug depot for delivering a therapeutic agent to a target tissue site beneath the skin of a patient is provided, the drug depot comprising an effective amount of a glucocorticoid, wherein the target tissue site comprises at least one muscle, ligament, tendon, cartilage, spinal disc, spinal foraminal space near the spinal nerve root, facet or synovial joint, or spinal canal.

In one exemplary embodiment, an implantable drug depot useful for reducing, preventing or treating adhesions in a patient in need of such treatment is provided, the implantable drug depot comprising a therapeutically effective amount of fluocinolone or dexamethasone or a pharmaceutically acceptable salt thereof, the depot being implantable at a site beneath the skin to reduce, prevent or treat adhesions, wherein the drug depot comprises (i) about 0.5 weight % to about 40 weight % of the fluocinolone or dexamethasone or a pharmaceutically acceptable salt thereof; (ii) about 60 weight % to about 99% of a polymer; and (iii) 1% to 50% of an excipient; where the drug depot is capable of releasing an effective amount of fluocinolone or dexamethasone or pharmaceutically acceptable salt thereof over a period of at least 1 week to 6 weeks. In various embodiments, the polymer comprises PLGA, DL-PLA, or a combination thereof and the excipient comprises mPEG, D-sorbitol, maltodextrin, PEG, cyclodextrin or a combination thereof.

In various embodiments, the drug depot comprises a gel, which includes a substance having a gelatinous, jelly-like, or colloidal properties at room temperature. The gel, in various embodiments, may have the glucocorticoid and optionally one or more additional therapeutic agents dispersed throughout it or suspended within the gel. The dispersal of the therapeutic agent may be even throughout the gel. Alternatively, the concentration of the therapeutic agent may vary throughout it. As the biodegradable material of the gel or drug depot degrades at the site, the therapeutic agent is released.

When the drug depot is a gel, in contrast to a sprayable gel that typically employs a low viscosity polymer, a gel with a higher viscosity may be desirable for other applications, for example, a gel having a putty-like consistency may be more preferable for adhesions near bone tissue.

In another exemplary embodiment, the gel is in viscous form is loaded with one or more drug depots (e.g., microspheres loaded with a therapeutic agent), wherein the viscous gel is positioned into a synovial joint, disc space, a spinal canal, or a soft tissue surrounding the spinal canal of a subject. The gel can also be used, in various embodiments, to seal or repair tissue as well as reduce, prevent or treat adhesions. In yet another exemplary embodiment, the gel is injectable, and/or an adherent gel that solidifies upon contact with tissue. For example, the gel may be administered as a liquid that gels in situ at the target tissue site. In various embodiments, the gel can comprise a two part system where a liquid is administered and a gelling agent is added subsequently to cause the liquid to gel or harden.

In various embodiments, the gel is a hardening gel, where after the gel is applied to the target site, it hardens and the drug can be released as the bodily fluid contacts the gel.

In various embodiments, the drug depot is loaded with a glucocorticoid and optionally one or more additional therapeutic agents, and delivered to the desired target tissue site (e.g., surgical wound site, inflammed tissue, degenerative tissue, etc.) and, in various embodiments, the drug depot may be held in place by a suture, barb, staple, adhesive gel, etc. which prevents the drug depot from being removed from that site by the venous systemic circulation or otherwise dispersed too widely, which reduces the desired therapeutic effect. For example, after hours or days, the drug depot may degrade, thereby allowing the drug depots (e.g., microspheres) to begin releasing the therapeutic agent. The microspheres may not begin releasing the agent until they are released from the drug depot. So, the microspheres may be formed from an insoluble or inert substances, but soluble or active once it comes into contact with the target tissue site. Likewise, the drug depot may comprise a substance that dissolves or disperses within the tissue. As the drug depot begins to dissolve within hours to days, the drug depots (e.g., microspheres) are exposed to body fluids and begin releasing their contents. The drug depot can be formulated to optimize exposure time of the drug depot and release of the therapeutic agent from the drug depot.

In various embodiments, the drug depot (e.g., gel) is flowable and can be injected, sprayed, instilled, and/or dispensed to, on or in the target tissue site. “Flowable” means that the gel formulation is easy to manipulate and may be brushed, sprayed, dripped, injected, shaped and/or molded at or near the target tissue site as it coagulates. “Flowable” includes formulations with a low viscosity or water-like consistency to those with a high viscosity, such as a paste-like material. In various embodiments, the flowability of the formulation allows it to conform to irregularities, crevices, cracks, and/or voids in the tissue site. For example, in various embodiments, the gel may be used to fill one or more voids in an osteolytic lesion.

In various embodiments, the drug depot comprises poly(alpha-hydroxy acids), poly(lactide-co-glycolide) (PLGA), polylactide (PLA), polyglycolide (PG), polyethylene glycol (PEG) conjugates of poly(alpha-hydroxy acids), polyorthoesters, polyaspirins, polyphosphagenes, collagen, starch, pre-gelatinized starch, hyaluronic acid, chitosans, gelatin, alginates, albumin, fibrin, vitamin E analogs, such as alpha tocopheryl acetate, d-alpha tocopheryl succinate, D,L-lactide, or L-lactide, -caprolactone, dextrans, vinylpyrrolidone, polyvinyl alcohol (PVA), PVA-g-PLGA, PEGT-PBT copolymer (polyactive), methacrylates, poly(N-isopropylacrylamide), PEO-PPO-PEO (pluronics), PEO-PPO-PAA copolymers, PLGA-PEO-PLGA, PEG-PLG (poly(d,l-lactide-co-glycolide), PLA-PLGA, poloxamer 407, PEG-PLGA-PEG triblock copolymers, SAIB (sucrose acetate isobutyrate) or combinations thereof. These one or more components allow the therapeutic agent to be released from the drug depot in a controlled and/or sustained manner. For example, the drug depot containing the therapeutic agent and a polymer matrix can be injected at the target tissue site and the polymer matrix breaks down over time (e.g., hours, days) within the target tissue site releasing a glucocorticoid and optionally additional therapeutic agents. Thus the administration of the drug depot can be localized and occur over a period of time (e.g., at least one day to about 1 to 8 weeks or longer).

The terms “sustained release” (e.g., extended release or controlled release) are used herein to refer to one or more therapeutic agent(s) that is introduced into the body of a human or other mammal and continuously releases a stream of one or more therapeutic agents over a predetermined time period and at a therapeutic level sufficient to achieve a desired therapeutic effect throughout the predetermined time period. Reference to a continuous release stream is intended to encompass release that occurs as the result of biodegradation in vivo of drug depot, or a matrix or component thereof, or as the result of metabolic transformation or dissolution of the therapeutic agent(s) or conjugates of therapeutic agent(s).

In various embodiments, the drug depot can be designed to cause an initial burst dose of therapeutic agent within the first 24 hours after implantation. “Initial burst” or “burst effect” or “bolus dose” refers to the release of therapeutic agent from the drug depot during the first 24 hours after the drug depot comes in contact with an aqueous fluid (e.g., synovial fluid, cerebral spinal fluid, etc.). In various embodiments, the drug depot is designed to avoid this initial burst effect.

In various embodiments, the drug depot contains one or more different release layer(s) that releases a bolus dose of a glucocorticoid or pharmaceutically acceptable salt thereof (e.g., 5 mg to 60 mg at a target site beneath the skin) and one or more sustain release layer(s) that releases an effective amount of a glucocorticoid or pharmaceutically acceptable salt thereof over a period of, for example, 1 to 8 weeks. In various embodiments, the one or more immediate release layer(s) comprise PLGA, which degrades faster and than the one or more sustain release layer(s), which comprises PLA, which degrades at a slower rate than the PLGA.

In various embodiments, when the drug depot comprises a gel, the gel may have a pre-dosed viscosity in the range of about 1 to about 500 centipoise (cps), 1 to about 200 cps, or 1 to about 100 cps. After the gel is administered to the target site, the viscosity of the gel will increase and the gel will have a modulus of elasticity (Young's modulus) in the range of about 1×10⁴ to about 6×10⁵ dynes/cm², or 2×10⁴ to about 5×10⁵ dynes/cm², or 5×10⁴ to about 5×10⁵ dynes/cm².

In one embodiment, the gel may be an adherent gel, which comprises a therapeutic agent that is evenly distributed throughout the gel. The gel may be of any suitable type, as previously indicated, and should be sufficiently viscous so as to prevent the gel from migrating from the targeted delivery site once deployed; the gel should, in effect, “stick” or adhere to the targeted tissue site. The gel may also adhere to the targeted tissue site not only by chemical processes, but by a mechanical interdigitation with the tissue prior to hardening.

The gel may, for example, solidify upon contact with the targeted tissue or after deployment from a targeted delivery system. The targeted delivery system may be, for example, a syringe, a catheter, needle or cannula or any other suitable device. The targeted delivery system may inject or spray the gel into or on the targeted tissue site. The therapeutic agent may be mixed into the gel prior to the gel being deployed at the targeted tissue site. In various embodiments, the gel may be part of a two-component delivery system and when the two components are mixed, a chemical process is activated to form the gel and cause it to stick or adhere to the target tissue.

In various embodiments, for those gel formulations that contain a polymer, the polymer concentration may affect the rate at which the gel hardens (e.g., a gel with a higher concentration of polymer may coagulate more quickly than gels having a lower concentration of polymer). In various embodiments, when the gel hardens, the resulting matrix is solid but is also able to conform to the irregular surface of the tissue (e.g., recesses and/or projections in bone).

The percentage of polymer present in the gel may also affect the viscosity of the polymeric composition. For example, a composition having a higher percentage by weight of polymer is typically thicker and more viscous than a composition having a lower percentage by weight of polymer. A more viscous composition tends to flow more slowly. Therefore, a composition having a lower viscosity may be preferred in some instances, for example when applying the formulation via spray.

In various embodiments, the molecular weight of the gel can be varied by many methods known in the art. The choice of method to vary molecular weight is typically determined by the composition of the gel (e.g., polymer, versus non-polymer). For example, in various embodiments, when the gel comprises one or more polymers, the degree of polymerization can be controlled by varying the amount of polymer initiators (e.g. benzoyl peroxide), organic solvents or activator (e.g. DMPT), crosslinking agents, polymerization agent, and/or reaction time.

Suitable gel polymers may be soluble in an organic solvent. The solubility of a polymer in a solvent varies depending on the crystallinity, hydrophobicity, hydrogen-bonding and molecular weight of the polymer. Lower molecular weight polymers will normally dissolve more readily in an organic solvent than high-molecular weight polymers. A polymeric gel, which includes a high molecular weight polymer, tends to coagulate or solidify more quickly than a polymeric composition, which includes a low-molecular weight polymer. Polymeric gel formulations, which include high molecular weight polymers, also tend to have a higher solution viscosity than a polymeric gel, which includes a low-molecular weight polymer.

In various embodiments, the gel can have a viscosity of about 300 to about 5,000 centipoise (cp). In other embodiments, the gel can have a viscosity of from about 5 to about 300 cps, from about 10 cps to about 50 cps, from about 15 cps to about 75 cps at room temperature, which allows it to be sprayed at or near the target site.

In various embodiments, the drug depot may comprise material to enhance viscosity and control the release of the drug. Such material may include, for example, hydroxypropyl cellulose, hydroxypropyl methylcellulose, hydroxyethyl methylcellulose, carboxymethylcellulose and salts thereof, Carbopol, poly(hydroxyethylmethacrylate), poly(methoxyethylmethacrylate), poly(methoxyethoxy-ethylmethacrylate), polymethyl-methacrylate (PMMA), methylmethacrylate (MMA), gelatin, polyvinyl alcohols, propylene glycol, PEG 200, PEG 300, PEG 400, PEG 500, PEG 550, PEG 600, PEG 700, PEG 800, PEG 900, PEG 1000, PEG 1450, PEG 3350, PEG 4500, PEG 8000 or combinations thereof. For example, in various embodiments, the drug depot comprises a polymer containing PLGA, DL-PLA, or a combination thereof and the excipient comprises mPEG, D-sorbitol, maltodextrin, 10% to 60% PEG 3350 MW, cyclodextrin or a combination thereof.

In various embodiments, the drug depot comprises poly(lactide-co-glycolide) (PLGA), polylactide (PLA), polyglycolide (PGA), D-lactide, D,L-lactide, L-lactide, D,L-lactide-ε-caprolactone, D,L-lactide-glycolide-ε-caprolactone, glycolide-caprolactone or a combination thereof.

In various embodiments, the gel has an inherent viscosity (abbreviated as “I.V.” and units are in deciliters/gram), which is a measure of the gel's molecular weight and degradation time (e.g., a gel with a high inherent viscosity has a higher molecular weight and longer degradation time). Typically, a gel with a high molecular weight provides a stronger matrix and the matrix takes more time to degrade. In contrast, a gel with a low molecular weight degrades more quickly and provides a softer matrix. In various embodiments, the gel has a molecular weight, as shown by the inherent viscosity, from about 0.10 dL/g to about 1.2 dL/g or from about 0.10 dL/g to about 0.40 dL/g. Other IV ranges include but are not limited to about 0.05 to about 0.15 dL/g, about 0.10 to about 0.20 dL/g, about 0.15 to about 0.25 dL/g, about 0.20 to about 0.30 dL/g, about 0.25 to about 0.35 dL/g, about 0.30 to about 0.35 dL/g, about 0.35 to about 0.45 dL/g, about 0.40 to about 0.45 dL/g, about 0.45 to about 0.50 dL/g, about 0.50 to about 0.70 dL/g, about 0.60 to about 0.80 dL/g, about 0.70 to about 0.90 dL/g, and about 0.80 to about 1.00 dL/g. In various embodiments, the drug depot may have an inherent viscosity as from about 0.05 to about 1.0 dL/g.

The drug depot release profile can also be controlled, among other things, by controlling the particle size distribution of the components of the drug depot. In various embodiments, the particle size distribution of the components of the drug depot (e.g., a glucocorticoid, gel, etc.) may be in the range of from about 10 μm to 100 μm so that the drug depot can easily be delivered to or at or near the target site by injection, spraying, instilling, etc.

In various embodiments, the drug depot may comprise a hydrogel made of high molecular weight biocompatible elastomeric polymers of synthetic or natural origin. A desirable property for the hydrogel to have is the ability to respond rapidly to mechanical stresses, particularly shears and loads, in the human body.

Hydrogels obtained from natural sources are particularly appealing since they are more likely to be biodegradable and biocompatible for in vivo applications. Suitable hydrogels include natural hydrogels, such as for example, gelatin, collagen, silk, elastin, fibrin and polysaccharide-derived polymers like agarose, and chitosan, glucomannan gel, hyaluronic acid, polysaccharides, such as cross-linked carboxyl-containing polysaccharides, or a combination thereof. Synthetic hydrogels include, but are not limited to those formed from polyvinyl alcohol, acrylamides such as polyacrylic acid and poly(acrylonitrile-acrylic acid), polyurethanes, polyethylene glycol (e.g., PEG 3350, PEG 4500, PEG 8000), silicone, polyolefins such as polyisobutylene and polyisoprene, copolymers of silicone and polyurethane, neoprene, nitrile, vulcanized rubber, poly(N-vinyl-2-pyrrolidone), acrylates such as poly(2-hydroxy ethyl methacrylate) and copolymers of acrylates with N-vinyl pyrolidone, N-vinyl lactams, polyacrylonitrile or combinations thereof. The hydrogel materials may further be cross-linked to provide further strength as needed. Examples of different types of polyurethanes include thermoplastic or thermoset polyurethanes, aliphatic or aromatic polyurethanes, polyetherurethane, polycarbonate-urethane or silicone polyether-urethane, or a combination thereof.

In various embodiments, rather than directly admixing the therapeutic agent into the drug depot, microspheres may be dispersed within the drug depot, the microspheres loaded with the therapeutic agent. In one embodiment, the microspheres provide for a sustained release of the therapeutic agent. In yet another embodiment, the drug depot, which is biodegradable, prevents the microspheres from releasing the therapeutic agent; the microspheres thus do not release the therapeutic agent until they have been released from the depot. For example, a drug depot may be deployed around a target tissue site (e.g., a nerve root). Dispersed within the drug depot are a plurality of microspheres that encapsulate the desired therapeutic agent. Certain of these microspheres degrade once released from the drug depot, thus releasing the therapeutic agent.

Microspheres, much like a fluid, may disperse relatively quickly, depending upon the surrounding tissue type, and hence disperse the therapeutic agent. In some situations, this may be desirable; in others, it may be more desirable to keep the therapeutic agent tightly constrained to a well-defined target site.

### Cannula or Needle

It will be appreciated by those with skill in the art that the depot can be administered to the target site using a cannula or needle that can be a part of a drug delivery device e.g., a syringe, a gun drug delivery device, or any medical device suitable for the application of a drug to a targeted organ or anatomic region. The cannula or needle of the drug depot device is designed to cause minimal physical and psychological trauma to the patient.

Cannulas or needles include tubes that may be made from materials, such as for example, polyurethane, polyurea, polyether(amide), PEBA, thermoplastic elastomeric olefin, copolyester, and styrenic thermoplastic elastomer, steel, aluminum, stainless steel, titanium, metal alloys with high non-ferrous metal content and a low relative proportion of iron, carbon fiber, glass fiber, plastics, ceramics or combinations thereof. The cannula or needle may optionally include one or more tapered regions. In various embodiments, the cannula or needle may be beveled. The cannula or needle may also have a tip style vital for accurate treatment of the patient depending on the site for implantation. Examples of tip styles include, for example, Trephine, Cournand, Veress, Huber, Seldinger, Chiba, Francine, Bias, Crawford, deflected tips, Hustead, Lancet, or Tuohey. In various embodiments, the cannula or needle may also be non-coring and have a sheath covering it to avoid unwanted needle sticks.

The dimensions of the hollow cannula or needle, among other things, will depend on the site for implantation. For example, the width of the epidural space is only about 3-5 mm for the thoracic region and about 5-7 mm for the lumbar region. Thus, the needle or cannula, in various embodiments, can be designed for these specific areas. In various embodiments, the cannula or needle may be inserted using a transforaminal approach in the spinal foramen space, for example, along an inflammed nerve root and the drug depot implanted at this site for treating the condition. Typically, the transforaminal approach involves approaching the intervertebral space through the intervertebral foramina.

Some examples of lengths of the cannula or needle may include, but are not limited to, from about 50 to 150 mm in length, for example, about 65 mm for epidural pediatric use, about 85 mm for a standard adult and about 110 mm for an obese adult patient. The thickness of the cannula or needle will also depend on the site of implantation. In various embodiments, the thickness includes, but is not limited to, from about 0.05 to about 1.655. The gauge of the cannula or needle may be the widest or smallest diameter or a diameter in between for insertion into a human or animal body. The widest diameter is typically about 14 gauge, while the smallest diameter is about 22 gauge. In various embodiments the gauge of the needle or cannula is about 18 to about 22 gauge.

In various embodiments, like the drug depot and/or gel, the cannula or needle includes dose radiographic markers that indicate location at or near the site beneath the skin, so that the user may accurately position the depot at or near the site using any of the numerous diagnostic imaging procedures. Such diagnostic imaging procedures include, for example, X-ray imaging or fluoroscopy. Examples of such radiographic markers include, but are not limited to, barium, calcium, and/or metal beads or particles.

In various embodiments, the needle or cannula may include a transparent or translucent portion that can be visualizable by ultrasound, fluoroscopy, x-ray, or other imaging techniques. In such embodiments, the transparent or translucent portion may include a radiopaque material or ultrasound responsive topography that increases the contrast of the needle or cannula relative to the absence of the material or topography.

The drug depot, and/or medical device to administer the drug may be sterilizable. In various embodiments, one or more components of the drug depot, and/or medical device to administer the drug are sterilized by radiation in a terminal sterilization step in the final packaging. Terminal sterilization of a product provides greater assurance of sterility than from processes such as an aseptic process, which require individual product components to be sterilized separately and the final package assembled in a sterile environment.

Typically, in various embodiments, gamma radiation is used in the terminal sterilization step, which involves utilizing ionizing energy from gamma rays that penetrates deeply in the device. Gamma rays are highly effective in killing microorganisms, they leave no residues nor have sufficient energy to impart radioactivity to the device. Gamma rays can be employed when the device is in the package and gamma sterilization does not require high pressures or vacuum conditions, thus, package seals and other components are not stressed. In addition, gamma radiation eliminates the need for permeable packaging materials.

In various embodiments, electron beam (e-beam) radiation may be used to sterilize one or more components of the device. E-beam radiation comprises a form of ionizing energy, which is generally characterized by low penetration and high-dose rates. E-beam irradiation is similar to gamma processing in that it alters various chemical and molecular bonds on contact, including the reproductive cells of microorganisms. Beams produced for e-beam sterilization are concentrated, highly-charged streams of electrons generated by the acceleration and conversion of electricity. E-beam sterilization may be used, for example, when the drug depot is included in a gel.

Other methods may also be used to sterilize the depot and/or one or more components of the device, including, but not limited to, gas sterilization, such as, for example, with ethylene oxide or steam sterilization.

In various embodiments, a kit is provided that may include additional parts along with the drug depot and/or medical device combined together to be used to implant the drug depot (e.g., ribbon-like fibers). The kit may include the drug depot device in a first compartment. The second compartment may include a canister holding the drug depot and any other instruments needed for the localized drug delivery. A third compartment may include gloves, drapes, wound dressings and other procedural supplies for maintaining sterility of the implanting process, as well as an instruction booklet. A fourth compartment may include additional cannulas and/or needles. Each tool may be separately packaged in a plastic pouch that is radiation sterilized. A cover of the kit may include illustrations of the implanting procedure and a clear plastic cover may be placed over the compartments to maintain sterility.

### Drug Delivery

In various embodiments, a method for delivering a glucocorticoid into a target tissue site of a patient is provided, the method comprising inserting a cannula at or near a target tissue site and implanting the drug depot containing a glucocorticoid at the target site beneath the skin of the patient. In various embodiments, to administer the drug depot to the desired site, first the cannula or needle can be inserted through the skin and soft tissue down to the target tissue site and the drug depot administered (e.g., injected, implanted, instilled, sprayed, etc.) at or near the target site. In those embodiments where the drug depot is separate from the gel, first the cannula or needle can be inserted through the skin and soft tissue down to the site of injection and one or more base layer(s) of gel can be administered to the target site. Following administration of the one or more base layer(s), the drug depot can be implanted on or in the base layer(s) so that the gel can hold the depot in place or reduce migration. If required a subsequent layer or layers of gel can be applied on the drug depot to surround the depot and further hold it in place. Alternatively, the drug depot may be implanted first and then the gel placed (e.g., brushed, dripped, injected, or painted, etc.) around the drug depot to hold it in place. By using the gel, accurate and precise implantation of a drug depot can be accomplished with minimal physical and psychological trauma to the patient. In various embodiments, the drug depot can be sutured to the target site or alternatively the drug depot can be implanted, without suturing. For example, in various embodiments, the drug depot can be a ribbon shaped depot and placed at the target site, before, during or after surgery.

In various embodiments, when the target tissue site comprises a spinal region, a portion of fluid (e.g., spinal fluid, etc.) can be withdrawn from the target site through the cannula or needle first and then the depot administered (e.g., placed, dripped, injected, or implanted, etc.). The target site will re-hydrate (e.g., replenishment of fluid) and this aqueous environment will cause the drug to be released from the depot.

“Localized” delivery includes, delivery where one or more drugs are deposited within, at or near a tissue. For example, localized delivery includes delivery to a nerve root of the nervous system or a region of the brain, or in close proximity (within about 0.1 cm to 10 cm, for example) thereto. “Targeted delivery system” provides delivery of one or more drugs depots (e.g., gels or depot dispersed in the gel, etc.) having a quantity of therapeutic agent that can be deposited at or near the target tissue site as needed for prevention, reduction, or treatment of adhesions.

### Adhesions

Adhesions are abnormal, fibrous bands of scar tissue that can form inside the body as a result of the healing process that often follows open or minimally invasive surgical procedure including abdominal, gynecologic, cardiothoracic, spinal, plastic, vascular, ENT, ophthalmologic, urologic, neuro, or orthopedic surgery. Adhesions are typically connective tissue structures that form between adjacent injured areas within the body. Briefly, localized areas of injury trigger an inflammatory and healing response that culminates in healing and scar tissue formation. If scarring results in the formation of fibrous tissue bands or adherence of adjacent anatomical structures (that should normally be separate), adhesion formation is said to have occurred.

Adhesions can range from flimsy, easily separable structures to dense, tenacious fibrous structures that can only be separated by surgical dissection. Adhesion-related complications may include, for example, small bowel obstruction, infertility, chronic pelvic pain or back pain, and other complications. Adhesions from a previous procedure can also complicate a second surgery, whether the surgery is planned or unexpected. In addition, the abnormal orientation of tissues and organs caused by adhesions may lead to further discomfort and chronic pain.

“Reducing adhesions” refers to administering a composition so as to cause a reduction in the number of adhesions, extent of adhesions (e.g., area), and/or severity of adhesions (e.g., thickness or resistance to mechanical or chemical disruption) relative to the number, extent, and/or severity of adhesions that would occur without such administration. In various embodiments, reducing adhesions may be part of a protocol and also include performing a procedure (e.g., subsequent surgery to reduce adhesions). The composition or procedure may inhibit formation, or growth of adhesions following an adhesion promoting stimulus, may inhibit progression of adhesions, and/or may inhibit recurrence of adhesions following their spontaneous regression or following mechanical or chemical disruption.

“Preventing adhesions” refers to administering a therapeutic composition prior to formation of adhesions in order to reduce the likelihood that adhesions will form in response to a particular insult, stimulus, or condition. In various embodiments, preventing adhesions may be part of a protocol and also include performing a procedure (e.g., surgery to reduce adhesions). It will be appreciated that “preventing adhesions” does not require that the likelihood of adhesion formation is reduced to zero. Instead, “preventing adhesions” refers to a clinically significant reduction in the likelihood of adhesion formation following a particular insult or stimulus, e.g., a clinically significant reduction in the incidence or number of adhesions in response to a particular adhesion promoting insult, condition, or stimulus.

“Treating adhesions,” refers to administering a composition that reverses, alleviates, reduces, and/or inhibits the progression and/or severity of adhesions, or reduces the likelihood of recurrence and/or the severity of recurrent adhesions. “Treating adhesions” also refers to administering or applying a composition that reverses, alleviates, reduces, inhibits the progression of, or reduces the likelihood of recurrence and/or severity of one or more symptoms of adhesions (e.g., pain, bowel obstruction, infertility, etc.). In various embodiments, treating adhesions may be part of a protocol and also include performing a procedure (e.g., surgery to reduce adhesions). Thus “treating adhesions” involves administering or applying a therapeutic composition and/or procedure once adhesion(s) have already formed following an insult or stimulus.

In various embodiments, an adhesion barrier may be administered or applied to the target tissue site before, during or with the drug depot to reduce, prevent or treat adhesions. Adhesion barriers work by separating opposing tissue surfaces or tissue-organ surfaces while injured tissues heal. Ingrowth of scar tissue and the formation or reformation of adhesions immediately adjacent to the barrier film is thus prevented.

One type of adhesion barrier is a thin film composed of chemically modified sugars, some of which occur naturally in the human body. The film adheres to tissues to which it is applied, and is slowly absorbed into the body over a period of about a week.

Another type of adhesion barrier is made of an amorphous bioresorbable copolymer, 70:30 Poly(L-lactide-co-D, L-lactide), which is designed to match the natural lactic acid produced in the body. As an inert material, the body accepts the polymer and processes it through the normal channels of bulk hydrolysis, followed by further breakdown in the liver into CO₂ and H₂O. Still another type of adhesion barrier based on PEG may be applied as two liquids, which are simultaneously sprayed onto the target area to form a soft adherent hydrogel. Within about one week, the hydrogel undergoes hydrolysis and is cleared from the body by the kidneys.

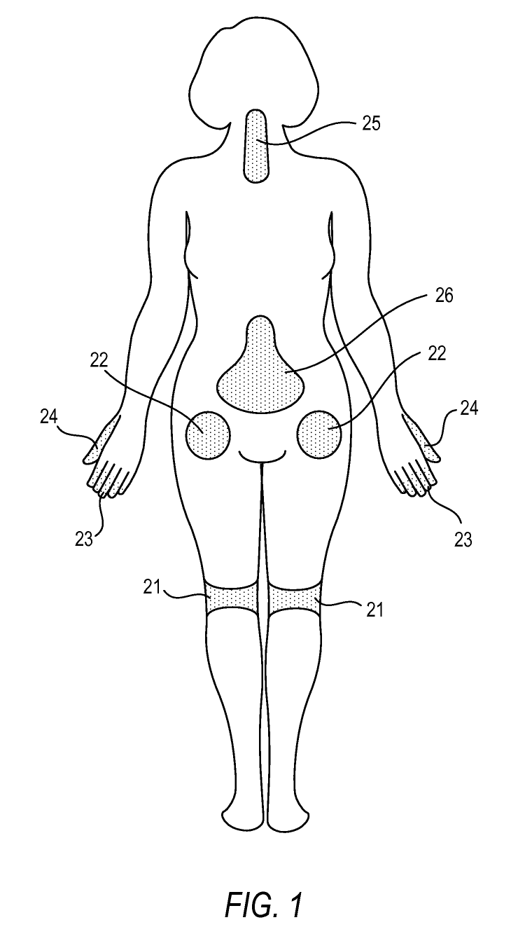

FIG. 1 illustrates a number of common locations within a patient that may be sites at which surgery took place and where adhesions may form. It will be recognized that the locations illustrated in FIG. 1 are merely exemplary of the many different locations within a patient that may be at which surgery took place and adhesions may form. For example, surgery may be required at a patient's knees 21, hips 22, fingers 23, thumbs 24, neck 25, and spine 26. Thus, during or following these surgeries, adhesions may form.

The term “pain” includes nociception and the sensation of pain, both of which can be assessed objectively and subjectively, using pain scores and other methods well-known in the art. In various embodiments, pain may include allodynia (e.g., increased response to a normally non-noxious stimulus) or hyperalgesia (e.g.,increased response to a normally noxious or unpleasant stimulus), which can in turn be thermal or mechanical (tactile) in nature. In some embodiments, pain is characterized by thermal sensitivity, mechanical sensitivity and/or resting pain. In other embodiments, pain comprises mechanically-induced pain or resting pain. In still other embodiments, the pain comprises resting pain. The pain can be primary or secondary pain, as is well-known in the art. Exemplary types of pain reducible, preventable or treatable by the methods and compositions disclosed herein include, without limitation, lower back pain, neck pain, leg pain, radicular pain, or abdominal pain from abdominal surgery, and neuropathic pain of the arm, neck, back, lower back, leg, and related pain distributions resulting from disk or spine surgery.

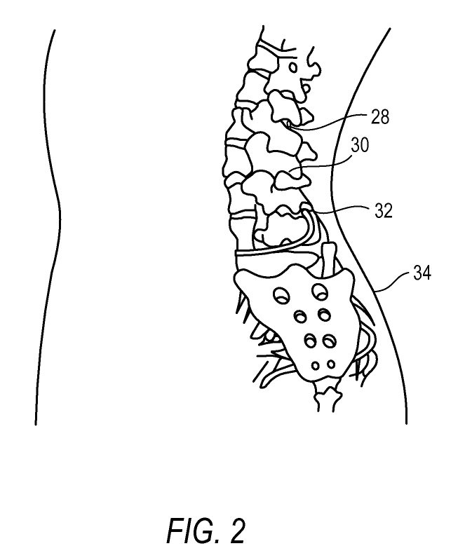

One exemplary embodiment where the depot is suitable for use to reduce, prevent or treat adhesions is illustrated in FIG. 2. Schematically shown in FIG. 2 is a dorsal view of the spine and sites where the drug depot may be inserted using a cannula or needle beneath the skin 34 to a spinal site 32 (e.g., spinal disc space, spinal canal, soft tissue surrounding the spine, nerve root, etc.) and one or more drug depots 28 and 32 are delivered to various sites along the spine. In this way, when several drug depots are to be implanted, they are implanted in a manner that optimizes location, accurate spacing, and drug distribution.

Although the spinal site is shown, as described above, the drug depot can be delivered to any site beneath the skin, including, but not limited to, at least one muscle, ligament, tendon, cartilage, spinal disc, spinal foraminal space, near the spinal nerve root, or spinal canal. In various embodiments, the drug depot containing a glucocorticoid can be administered to the patient intra-operatively, intravenously, intramuscularly, SC, intrathecally, intradiskally, peridiskally, epidurally, perispinally, or parenterally or combinations thereof.

The term “patient” refers to organisms from the taxonomy class “mammalian,” including, but not limited to, humans, other primates such as chimpanzees, apes orangutans and monkeys, rats, mice, cats, dogs, cows, horses, etc.

### Method of Making Glucocorticoid Depots

In various embodiments, the drug depot comprising the a glucocorticoid can be made by combining a biocompatible polymer and a therapeutically effective amount of a glucocorticoid or pharmaceutically acceptable salt thereof and forming the implantable drug depot from the combination.

Various techniques are available for forming at least a portion of a drug depot from the biocompatible polymer(s), therapeutic agent(s), and optional materials, including solution processing techniques and/or thermoplastic processing techniques. Where solution processing techniques are used, a solvent system is typically selected that contains one or more solvent species. The solvent system is generally a good solvent for at least one component of interest, for example, biocompatible polymer and/or therapeutic agent. The particular solvent species that make up the solvent system can also be selected based on other characteristics, including drying rate and surface tension.

Solution processing techniques include solvent casting techniques, spin coating techniques, web coating techniques, solvent spraying techniques, dipping techniques, techniques involving coating via mechanical suspension, including air suspension (e.g., fluidized coating), ink jet techniques and electrostatic techniques. Where appropriate, techniques such as those listed above can be repeated or combined to build up the depot to obtain the desired release rate and desired thickness.

In various embodiments, a solution containing solvent and biocompatible polymer are combined and placed in a mold of the desired size and shape. In this way, polymeric regions, including barrier layers, lubricious layers, and so forth can be formed. If desired, the solution can further comprise, one or more of the following: a glucocorticoid and other therapeutic agent(s) and other optional additives such as radiographic agent(s), etc. in dissolved or dispersed form. This results in a polymeric matrix region containing these species after solvent removal. In other embodiments, a solution containing solvent with dissolved or dispersed therapeutic agent is applied to a pre-existing polymeric region, which can be formed using a variety of techniques including solution processing and thermoplastic processing techniques, whereupon the therapeutic agent is imbibed into the polymeric region.

Thermoplastic processing techniques for forming the depot or portions thereof include molding techniques (for example, injection molding, rotational molding, and so forth), extrusion techniques (for example, extrusion, co-extrusion, multi-layer extrusion, and so forth) and casting.

Thermoplastic processing in accordance with various embodiments comprises mixing or compounding, in one or more stages, the biocompatible polymer(s) and one or more of the following: a glucocorticoid, optional additional therapeutic agent(s), radiographic agent(s), and so forth. The resulting mixture is then shaped into an implantable drug depot. The mixing and shaping operations may be performed using any of the conventional devices known in the art for such purposes.

During thermoplastic processing, there exists the potential for the therapeutic agent(s) to degrade, for example, due to elevated temperatures and/or mechanical shear that are associated with such processing. For example, a glucocorticoid tromethamine may undergo substantial degradation under ordinary thermoplastic processing conditions. Hence, processing is preferably performed under modified conditions, which prevent the substantial degradation of the therapeutic agent(s). Although it is understood that some degradation may be unavoidable during thermoplastic processing, degradation is generally limited to 10% or less. Among the processing conditions that may be controlled during processing to avoid substantial degradation of the therapeutic agent(s) are temperature, applied shear rate, applied shear stress, residence time of the mixture containing the therapeutic agent, and the technique by which the polymeric material and the therapeutic agent(s) are mixed.

Mixing or compounding biocompatible polymer with therapeutic agent(s) and any additional additives to form a substantially homogenous mixture thereof may be performed with any device known in the art and conventionally used for mixing polymeric materials with additives.

Where thermoplastic materials are employed, a polymer melt may be formed by heating the biocompatible polymer, which can be mixed with various additives (e.g., therapeutic agent(s), inactive ingredients, etc.) to form a mixture. A common way of doing so is to apply mechanical shear to a mixture of the biocompatible polymer(s) and additive(s). Devices in which the biocompatible polymer(s) and additive(s) may be mixed in this fashion include devices such as single screw extruders, twin screw extruders, banbury mixers, high-speed mixers, ross kettles, and so forth.

Any of the biocompatible polymer(s) and various additives may be premixed prior to a final thermoplastic mixing and shaping process, if desired (e.g., to prevent substantial degradation of the therapeutic agent among other reasons).

For example, in various embodiments, a biocompatible polymer is precompounded with a radiographic agent (e.g., radio-opacifying agent) under conditions of temperature and mechanical shear that would result in substantial degradation of the therapeutic agent, if it were present. This precompounded material is then mixed with therapeutic agent under conditions of lower temperature and mechanical shear, and the resulting mixture is shaped into the glucocorticoid containing drug depot. Conversely, in another embodiment, the biocompatible polymer can be precompounded with the therapeutic agent under conditions of reduced temperature and mechanical shear. This precompounded material is then mixed with, for example, a radio-opacifying agent, also under conditions of reduced temperature and mechanical shear, and the resulting mixture is shaped into the drug depot.

The conditions used to achieve a mixture of the biocompatible polymer and therapeutic agent and other additives will depend on a number of factors including, for example, the specific biocompatible polymer(s) and additive(s) used, as well as the type of mixing device used.

As an example, different biocompatible polymers will typically soften to facilitate mixing at different temperatures. For instance, where a depot is formed comprising PLGA or PLA polymer, a radio-opacifying agent (e.g., bismuth subcarbonate), and a therapeutic agent prone to degradation by heat and/or mechanical shear (e.g., a glucocorticoid), in various embodiments, the PGLA or PLA can be premixed with the radio-opacifying agent at temperatures of about, for example, 150° C. to 170° C. The therapeutic agent is then combined with the premixed composition and subjected to further thermoplastic processing at conditions of temperature and mechanical shear that are substantially lower than is typical for PGLA or PLA compositions. For example, where extruders are used, barrel temperature, volumetric output are typically controlled to limit the shear and therefore to prevent substantial degradation of the therapeutic agent(s). For instance, the therapeutic agent and premixed composition can be mixed/compounded using a twin screw extruder at substantially lower temperatures (e.g., 100-105° C.), and using substantially reduced volumetric output (e.g., less than 30% of full capacity, which generally corresponds to a volumetric output of less than 200 cc/min). It is noted that this processing temperature is well below the melting points of the glucocorticoid, because processing at or above these temperatures will result in substantial therapeutic agent degradation. It is further noted that in certain embodiments, the processing temperature will be below the melting point of all bioactive compounds within the composition, including the therapeutic agent. After compounding, the resulting depot is shaped into the desired form, also under conditions of reduced temperature and shear.

In other embodiments, biodegradable polymer(s) and one or more therapeutic agents are premixed using non-thermoplastic techniques. For example, the biocompatible polymer can be dissolved in a solvent system containing one or more solvent species. Any desired agents (for example, a radio-opacifying agent, a therapeutic agent, or both radio-opacifying agent and therapeutic agent) can also be dissolved or dispersed in the solvents system. Solvent is then removed from the resulting solution/dispersion, forming a solid material. The resulting solid material can then be granulated for further thermoplastic processing (for example, extrusion) if desired.

As another example, the therapeutic agent can be dissolved or dispersed in a solvent system, which is then applied to a pre-existing drug depot (the pre-existing drug depot can be formed using a variety of techniques including solution and thermoplastic processing techniques, and it can comprise a variety of additives including a radio-opacifying agent and/or viscosity enhancing agent), whereupon the therapeutic agent is imbibed on or in the drug depot. As above, the resulting solid material can then be granulated for further processing, if desired.

Typically, extrusion processes may be used to form the drug depot comprising the biocompatible polymer(s), therapeutic agent(s) and radio-opacifying agent(s). Co-extrusion may also be employed, which is a shaping process that can be used to produce a drug depot comprising the same or different layers or regions (for example, a structure comprising one or more polymeric matrix layers or regions that have permeability to fluids to allow immediate and/or sustained drug release). Multi-region depots can also be formed by other processing and shaping techniques such as co-injection or sequential injection molding technology.

In various embodiments, the depot that may emerge from the thermoplastic processing (e.g., ribbon, pellet, strip, etc.) is cooled. Examples of cooling processes include air cooling and/or immersion in a cooling bath. In some embodiments, a water bath is used to cool the extruded depot. However, where a water-soluble therapeutic agent such as a glucocorticoid is used, the immersion time should be held to a minimum to avoid unnecessary loss of therapeutic agent into the bath.

In various embodiments, immediate removal of water or moisture by use of ambient or warm air jets after exiting the bath will also prevent re-crystallization of the drug on the depot surface, thus controlling or minimizing a high drug dose “initial burst” or “bolus dose” upon implantation or insertion if this is release profile is not desired.

In various embodiments, the drug depot can be prepared by mixing or spraying the drug with the polymer and then molding the depot to the desired shape. In various embodiments, a glucocorticoid is used and mixed or sprayed with the polymer, and the resulting depot may be formed by extrusion and dried.

### EXAMPLES

Certain abbreviations are used in the tables and figures. The abbreviation “DLG” refers to poly(DL-lactide-co-glycolide). The abbreviation “DL” refers to poly(DL-lactide). The abbreviation “LG” refers to poly(L-lactide-co-glycolide). The abbreviation “CL” refers to polycaprolactone. The abbreviation “DLCL” refers to poly(DL-lactide-co-caprolactone). The abbreviation “LCL” refers to poly(L-lactide-co-caprolactone). The abbreviation “G” refers to polyglycolide. The abbreviation “PEG” refers to poly(ethylene glycol). The abbreviation “PLGA” refers to poly(lactide-co-glycolide). The abbreviation “PLA” refers to polyglycolide. “POE” refers to poly(ortho ester). “SAIB” refers to sucrose acetate isobutyrate, which is a water insoluble biodegradable gel used for sustained release properties.

Often times when the polymer is a heteropolymer or copolymer, there is a mixture of monomer species in the polymer. The mole ratio may be indicated and varied from 0:100 to 100:0 and ranges in between these mole ratios. For example, 85:15 DLG, the 85 refers to the monomer mole % 85 of DL (poly DL-lactide) in the polymer, while the 15 refers to the mole percent of the G (polyglycolide) in the polymer.

### Example 1

The behavioral animal model of chronic constriction injury (“CCI”) was chosen to evaluate the efficacy of steroids and NSAIDS on pain treatment. This model may mimic pain associated with sciatica in humans.

Surgical Procedures

Wister rats (Charles River Laboratories, Wilmington, Mass.) weighing 300±26 g the day of surgery (Day 1) were used in this study. All experiments were conducted in accordance with the International Association for the Study of Pain guidelines and approved by the Institutional Animal Care and Use Committee at SRI International, Inc (Menlo Park, Calif.). CCI was induced according to the method of Bennett and Xie. Briefly, each animal was anesthetized by intraperitoneal (IP) injection of sodium pentobarbital at a dose of 60 mg/kg. The animal's common sciatic nerve was exposed and freed from adherent tissue at mid-thigh by separating the biceps femoris muscles by blunt dissection. Four loose ligatures were placed 1 mm apart, using chromic gut suture (4-0 absorbable suture; Jorgensen Laboratories, Inc., Loveland, Colo.).

### Example 2

Task 31 Explant Notes:

A 2-month chronic constriction injury (CCI) model of neuropathic pain was used to evaluate different formulations of a corticosteroid (fluocinolone) encapsulated in bioerodible polymers. Different formulations were evaluated for reducing pain-associated behaviors. Thermal hyperalgesia was evaluated at 2, 7, 14, 21, 28, 35, 42, 49, and 56 days post-operatively, while mechanical allodynia was evaluated at 1, 8, 15, 22, 29, 36, 43, 50, and 57 days post-operatively.

There were seven groups of animals tested. Each animal received treatment of test or control article according to the dosing groups (n=8) described in the Table A. Group 1 received daily drug injections. Groups 2-7 received solid, polymer implants that were implanted caudal to the CCI in a manner that totally surrounds the nerve.

TABLE A

| Group | | | |
|---|---|---|---|
| Number | Treatment | Dose | Comments |
| 1 | Fluocinolone | 0.5 | Daily SC administration |
| | | ug/kg | |
| 2 | 85/15 PLGA | 0% | control |
| 3 | 100 DL 5E + Glacial acetic | 0% | control |
| | acid/7% 5050 7A | | |
| 4 | 85/15 PLGA + 10% mPEG | 20% | 1 pellet |
| 5 | 100 DL 5E + 10% PEG1500 | 1% | 1 pellet |
| 6 | 100 DL 7E + 5% PEG/7% | 1% | 1 pellet |
| | 5050 7A | | |
| 7 | 100 DL 7E + 10% PEG1500 | 1% | 1 pellet |

### Study Duration: 57 Days

One week following the last behavioral test, the animals were sacrificed, and the residual polymer depots were removed for additional analysis. The following are observations made during the explant procedure.

Group 1=systemic injection group sacrificed after last round of behavioral testing.
Group 2=white pellets, no noticeable tissue reactions, moderate encapsulation.
Group 3=white pellets, no noticeable tissue reactions, moderate encapsulation.
Group 4=small, white pellet; no noticeable tissue reactions; little to no encapsulation (pellets appeared as if just sitting in the tissue); sutures appear relatively intact. Animal 2=no retrievable pellet; major suture granuloma.
Group 5=small, white pellet; no noticeable tissue reactions; little to no encapsulation
Group 6=small, white pellet; no noticeable tissue reactions; little to no encapsulation; sutures appear relatively intact.
Group 7=small, white pellet; no noticeable tissue reactions; little to no encapsulation

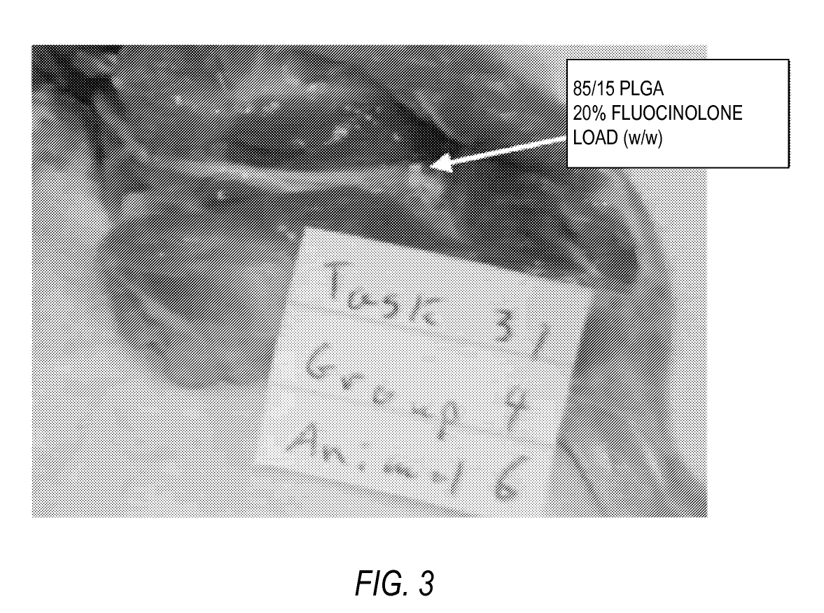

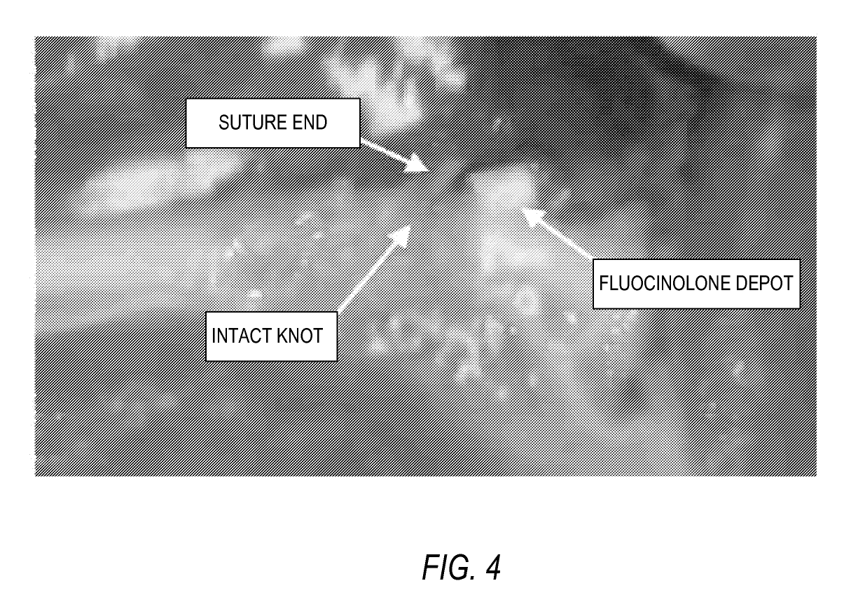

FIGS. 3 and 4 show pictures of the surgical wound of an animal in Group 4 where the following was noted (i) no noticeable tissue reactions occurred; (ii) little to no encapsulation (pellets appeared as if just sitting in the tissue); and (iii) sutures appear relatively intact by the common sciatic nerve. No adhesions formation was noted after 57 days, which was remarkable. The drug depot contained 20 wt % fluocinolone, 70 wt % 85/15 and PLGA 10 wt % mPEG and the pellet size was 0.75 mm×0.75 mm,

### Example 3

Task 30: Evaluating the Efficacy of a 5 Month Polymer Drug Depot in the Rat Chronic Constriction Injury Model

A 2-month chronic constriction injury (CCI) model of neuropathic pain was used to evaluate different formulations of the NSAID sulindac encapsulated in a bioerodible polymer, poly(lactic-co-glycolic acid). Different formulations were evaluated for reducing pain-associated behaviors. Thermal hyperalgesia was evaluated at 2, 7, 14, 21, 28, 35, 42, 49, and 56 days post-operatively, while mechanical allodynia was evaluated at 1, 8, 15, 22, 29, 36, 43, 50, and 57 days post-operatively.

Experimental Design: Four loose chromic gut ligatures, 1 mm apart, were tied around the common sciatic nerve at mid-thigh. Each animal received treatment of test or control articles according to the dosing groups (n=7) described in Table B. Group 1 received daily drug injections as indicated. Groups 2-8 received solid, polymer implants that were implanted caudal to the CCI in a manner that totally surrounds the nerve.

TABLE B

| Group | | | |
|---|---|---|---|
| Number | Treatment | Dose | Comments |
| 1 | Sulindac | 0.4 mg/kg | Daily IP administration |
| 2 | 85/15 PLGA + 10% | — | Control |
| | mPRG | | |
| 3 | 85/15 PLGA + 10% | 20% Load | Six 0.75 mm × 4.0 mm |
| | mPEG | | pellets |
| 4 | 85/15 PLGA + 10% | 27% Load | Six 0.75 mm × 4.0 mm |
| | mPEG | | pellets |
| 5 | DL-PLA + 10% mPEG | 25% Load | Six 0.75 mm × 4.0 mm |
| | | | pellets |
| 6 | 100 DL 7E | 30% load | 3 implants |
| 7 | 100 DL 7E | 20% load | 6 implants |
| 8 | 85/15 PLGA | 30% load | 3 implants |

### Study Duration: 57 Days

One week following the last behavioral test, the animals were sacrificed and the residual polymer depots were removed for additional analysis. The following are observations made during the explant procedure.

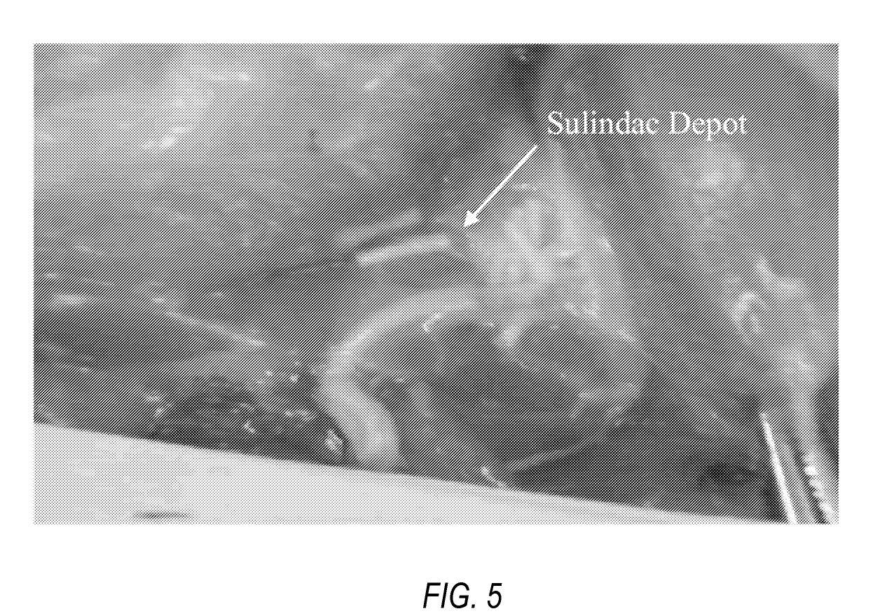

The depot containing sulindac (an NSAID) showed scar tissue encapsulation around the sulindac depots (pellets). FIG. 5 shows a picture of the surgical wound and the scar tissue and fibrous tissue formed in the sulindac treated group.

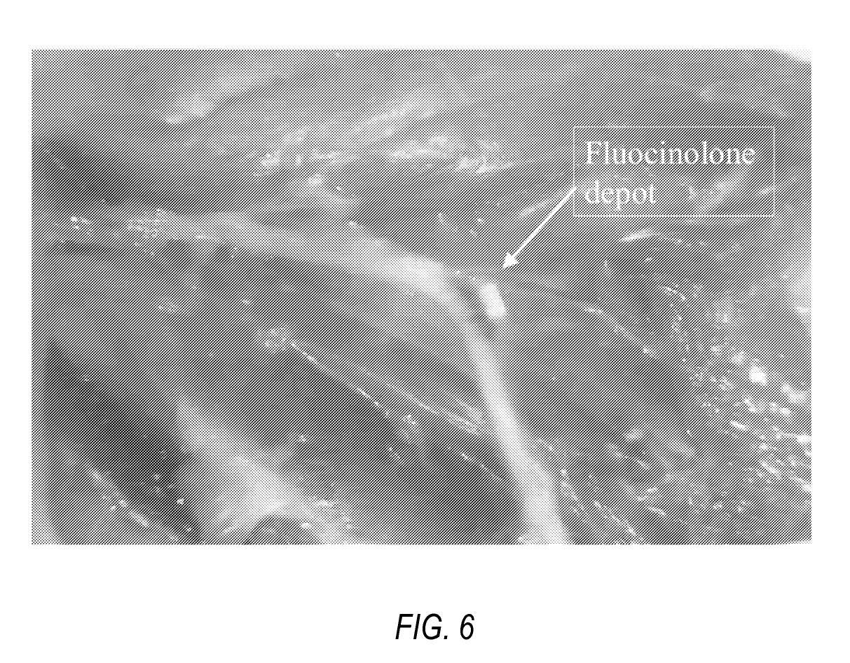

FIG. 6 shows a picture of the surgical wound after 65 days and the drug depot containing 1 wt % fluocinolone, 100 DL+5% PEG/7% 50/50 PLGA. Note less scar tissue and no adhesions were formed.

### Example 4

Fluocinolone Formulations and Release Profiles

Fluocinolone is a potent steroid with glucocorticoid activity. To get consistent release additional excipients were added to the polymer formulation. For example with drug loads of 1% to 20% fluocinolone, 85/15 PLGA or DL-PLA or DL-PLA and 50/50 PLGA mixtures can be added in an amount of from about 10% to 98%. The depot can be extruded and made into different sizes (e.g., 0.75 (length)×0.75 mm (diameter), 0.8×0.8 mm, 1×1 mm pellet sizes, etc.).

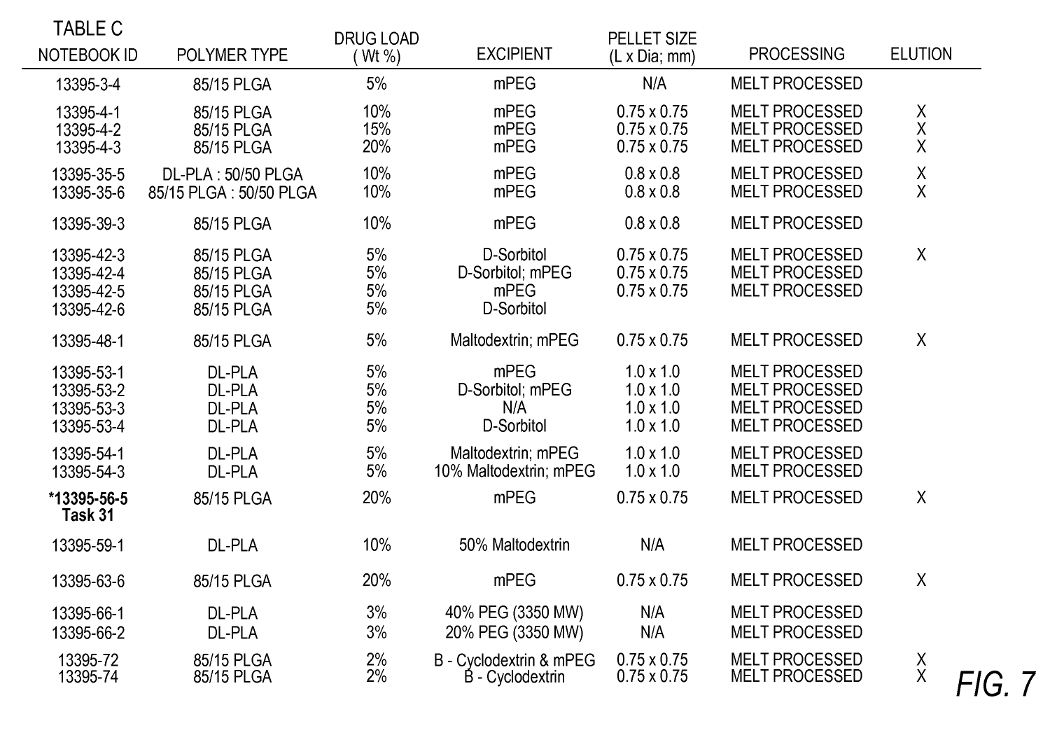

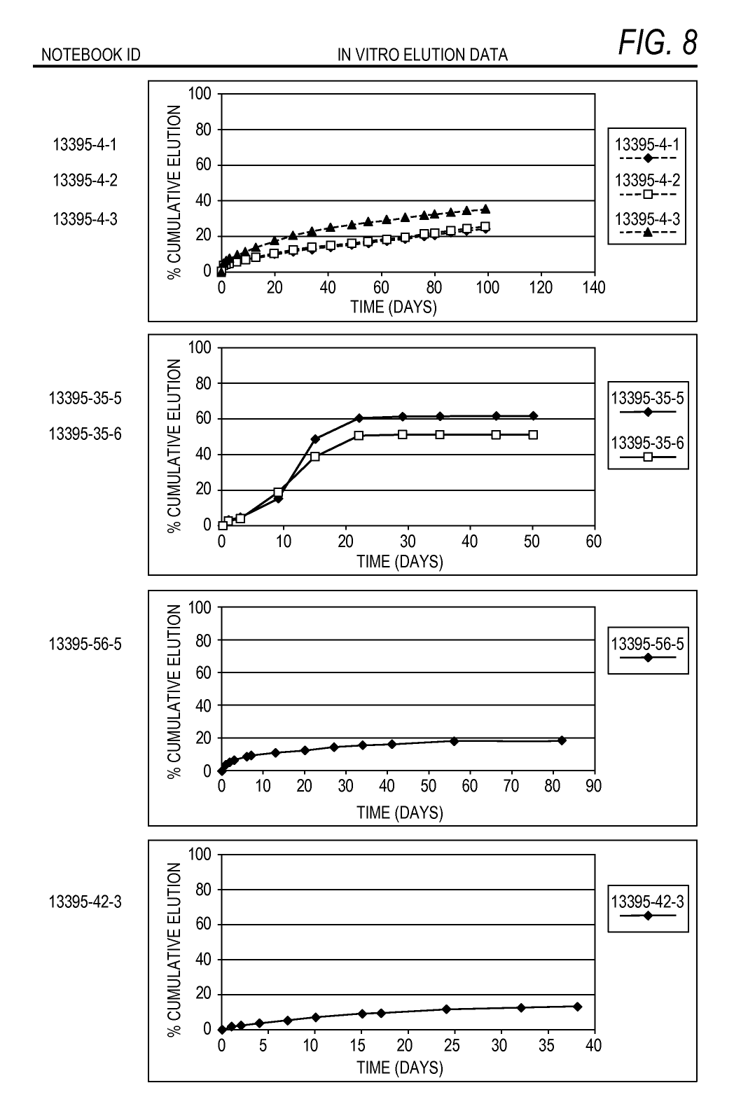

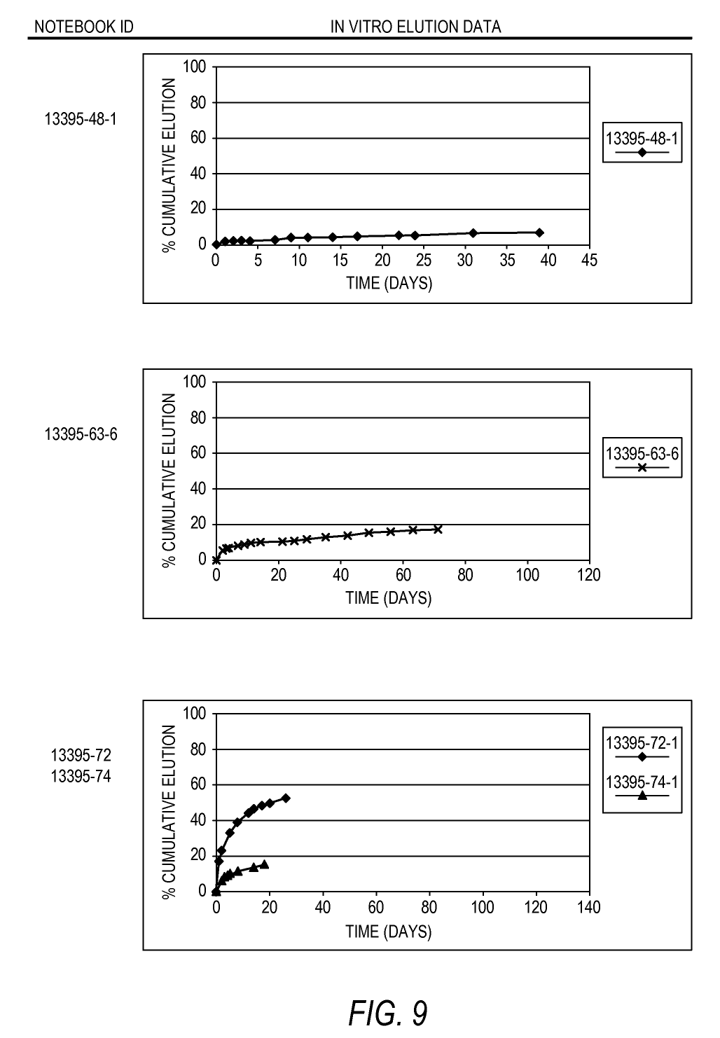

FIG. 7 (Table C) shows various fluocinolone formulations made and some of their in vitro elution profiles ranging from about 20 days to about 100 days with fluocinolone release rates starting at 0% to 60% cumulative release shown in FIGS. 8 and 9. The in-vitro elution studies were carried out at 37° C. in phosphate-buffered saline (PBS) with 0.5% SDS (pH 7.4). Briefly, the rods (n=3) were weighed prior to immersion in 10 mL of PBS. At regular time intervals, the PBS was removed for analysis and replaced with 10 mL of fresh PBS. The PBS-elution buffer was analyzed for fluocinolone content using UV-Vis spectrometry.

It was noted that the fluocinolone depot discussed in Example 2 above had an almost linear release of fluocinolone in the animal that had no adhesion formation elution profile shown in 13395-56-5 in FIG. 8.

It will be apparent to those skilled in the art that various modifications and variations can be made to various embodiments described herein without departing from the spirit or scope of the teachings herein. Thus, it is intended that various embodiments cover other modifications and variations of various embodiments within the scope of the present teachings.

What is claimed is:

1. A method of reducing, preventing or treating adhesions in a patient in need of such treatment, the method comprising administering one or more biodegradable drug depots comprising a therapeutically effective amount of a glucocorticoid or a pharmaceutically acceptable salt thereof at or near a target tissue site beneath the skin, wherein the one or more biodegradable drug depot is capable of releasing an effective amount of the glucocorticoid or pharmaceutically acceptable salt thereof over a period of at least 1 day to 6 months.

2. A method according to claim 1, wherein the one or more drug depots prevent post operative surgical adhesions by releasing a therapeutically effective amount of the glucocorticoid to prevent in-growth of scar tissue and formation or reformation of adhesions at or near the target tissue site while an injured tissue heals.

3. A method according to claim 1, wherein the glucocorticoid comprises fluocinolone or dexamethasone or a pharmaceutically acceptable salt thereof.

4. A method according to claim 3, wherein the one or more drug depots release an effective amount of the fluocinolone or dexamethasone or a pharmaceutically acceptable salt thereof over a period of at least 1 week to 6 weeks.

5. A method according to claim 4, wherein the one or more drug depot releases 0.05 mcg to 2 mcg of fluocinolone or pharmaceutically acceptable salt thereof every 24 hours for a period of at least 1 to 6 weeks.

6. A method according to claim 1, wherein the one or more drug depot release 5%, 10%, 15%, 20%, 25%, 30%, 40%, 50%, 60%, 70%, 80%, 90%, 95%, or 99% of the glucocorticoid or a pharmaceutically acceptable salt thereof relative to a total amount of glucocorticoid loaded in the drug depot over a period of at least 1 week to 6 weeks after the drug depot is administered to the target tissue site.

7. A method according to claim 1, wherein the one or more drug depot is loaded with a glucocorticoid in an amount of about 0.5% to 40% by weight based on the total weight of the drug depot.

8. A method according to claim 1, wherein the target tissue site comprises at least one muscle, ligament, tendon, cartilage, spinal disc, spinal foraminal space near the spinal nerve root, facet or synovial joint, or spinal canal.

9. A method according to claim 1, wherein the glucocorticoid or pharmaceutically acceptable salt thereof is encapsulated in a plurality of depots comprising microparticles, microspheres, microcapsules, and/or microfibers suspended in a gel.

10. A method according to claim 1, wherein the one or more drug depot is delivered to the target tissue site beneath the skin before, during or after surgery.

11. A method according to claim 1, wherein, a barrier is administered before, after or with the one or more drug depots at or near the target tissue site.

12. A method of according to claim 1, wherein the glucocorticoid comprises fluocinolone acetonide, fluocinonide, dexamethasone, dexamethasone sodium phosphate, dexamethasone acetate or a pharmaceutically acceptable salt thereof or a combination thereof.

13. A method of according to claim 1, wherein the glucocorticoid comprises cortisone, hydrocortisone, prednisone, prednisolone, methylprednisolone, budesonide, dexamethasone, fludrocortisone, fluocortolone, cloprednole, deflazacort, triamcinolone, or a pharmaceutically acceptable salt thereof or combination thereof.

14. A method according to claim 1, wherein the drug depot comprises a polymer comprising poly(lactide-co-glycolide) (PLGA), polylactide (PLA), polyglycolide (PGA), D-lactide, D,L-lactide, L-lactide, D,L-lactide-caprolactone, D,L-lactide-glycolide-caprolactone or a combination thereof.

15. An implantable drug depot useful for reducing, preventing or treating adhesions in a patient in need of such treatment, the implantable drug depot comprising a therapeutically effective amount of a glucocorticoid or a pharmaceutically acceptable salt thereof, the depot being implantable at a site beneath the skin to reduce, prevent or treat adhesions, wherein the drug depot is loaded with about 0.5 weight % to about 40 weight % of the glucocorticoid or a pharmaceutically acceptable salt thereof and is capable of releasing an effective amount of a glucocorticoid or pharmaceutically acceptable salt thereof over a period of at least 1 week to 6 weeks.

16. An implantable drug depot according to claim 15, wherein the drug depot comprises fluocinolone acetonide, fluocinonide, dexamethasone, dexamethasone sodium phosphate dexamethasone acetate or a pharmaceutically acceptable salt thereof or a combination thereof.

17. An implantable drug depot according to claim 15, wherein the glucocorticoid comprises fluocinolone or dexamethasone or a pharmaceutically acceptable salt thereof.

18. An implantable drug depot according to claim 15, wherein the drug depot comprises a polymer comprising poly(lactide-co-glycolide) (PLGA), polylactide (PLA), polyglycolide (PGA), D-lactide, D,L-lactide, L-lactide, D,L-lactide-caprolactone, D,L-lactide-glycolide-caprolactone or a combination thereof.

19. An implantable drug depot according to claim 18, wherein the polymer comprises from about 10% to about 99% of the total weight % of the drug depot.

20. A method of making an implantable drug depot of claim 15, the method comprising combining a biocompatible polymer and a therapeutically effective amount of a glucocorticoid or a pharmaceutically acceptable salt thereof and forming the implantable drug depot from the combination.
